(19) United States
(12) Patent Application Publication
(10) Pub. No.: US 2020/0221703 A1
(43) Pub. Date: Jul. 16, 2020

(54) BACTERIAL STRAINS AND THEIR USE FOR CONTROLLING PLANT DISEASE

(71) Applicant: AgBiome, Inc., Durham, NC (US)

(72) Inventors: Philip E. Hammer, Cary, NC (US); Mark A. Roberts, Chapel Hill, NC (US); Mathias Twizeyimana, Durham, NC (US); Steve Ronyak, Durham, NC (US)

(73) Assignee: AgBiome, Inc., Durham, NC (US)

(21) Appl. No.: 16/746,109

(22) Filed: Jan. 17, 2020

Related U.S. Application Data
(63) Continuation of application No. 15/759,660, filed on Mar. 13, 2018, now abandoned.
(60) Provisional application No. 62/211,282, filed on Aug. 28, 2015.
(60) Provisional application No. 62/320,840, filed on Apr. 11, 2016.

(51) Int. Cl. A01N 63/00; C12R 1/01; C12R 1/125; C12N 1/20; C12R 1/07; A01N 25/14

(52) U.S. Cl. CPC A01N 63/00; C12R 1/01; C12R 1/125; A01N 25/14; C12R 1/07; C12R 1/075; C12N 1/20

(57) ABSTRACT

Compositions and methods for treating or preventing plant disease are provided. Such compositions and methods comprise a bacterial strain that control one or more pathogens that cause plant disease or improve at least one agronomic trait of interest in a plant. The bacterial strain can be used as an inoculant for plants. Therefore, methods for growing a plant susceptible to a plant disease and methods for controlling plant disease on a plant susceptible to the plant disease are provided.

This application claims the benefit of U.S. Provisional Application No. 62/320,840, filed Apr. 11, 2016, and U.S. Provisional Application No. 62/211,282, filed Aug. 28, 2015, both of which are hereby incorporated herein in their entireties by this reference.

### FIELD OF THE INVENTION

The invention relates to bacterial strains and populations for controlling plant disease and/or improving an agronomic trait of interest in a plant.

### BACKGROUND

Plant diseases are responsible for significant agricultural losses. Effects can range from mild symptoms to catastrophic plant damage, which can lead to major economic and social consequences. Methods are needed to effectively control plant diseases and the pathogens that cause them.

### SUMMARY

Compositions and methods for controlling plant disease and/or for improving at least one agronomic trait of interest in a plant are provided. Such compositions and methods comprise a population of biocontrol agents or bacterial strains that control one or more pathogens that cause plant disease and/or improve at least one agronomic trait of interest. The biological agents or bacterial strains can be used as an inoculant for plants. Methods for growing a plant susceptible to plant disease and methods and compositions for controlling plant disease are also provided. Further provided are methods and compositions of increasing disease resistance in plants. Methods and compositions for improving plant health and/or improving at least one agronomic trait of interest are also provided.

### BRIEF DESCRIPTION OF FIGURES

FIG. 1 shows inhibition of Asian soybean rust (ASR) development on whole plant in growth chambers by different bacterial strains. Fungicide azoxystrobin and (+) control strain were added as positive controls while AFS006 and inoculated control were negative controls.

FIG. 2 shows the number of germinated seedlings (stand count) per acre by bacterial strains AIP061892 and AIP079428. This figure demonstrates that AIP061892 and AIP079428 produced about a 2-fold increase in germination over Pythium inoculated control.

FIG. 3 shows the number of germinated seedlings (stand count) per acre by bacterial strain AIP061892 and AIP079428. This figure demonstrates that AIP061892 produced a 50% recovery in germination over Rhizoctonia solani inoculated control.

### DETAILED DESCRIPTION

I. Overview Compositions and methods for improving at least one agronomic trait of interest and/or improving plant health and/or for controlling one or more plant diseases are provided. A biological agent, biocontrol agent, bacterial strain, modified bacterial strain, modified biological agent, or modified biocontrol agent or active variant thereof are used herein to describe a microorganism that is used to control disease-causing plant pathogens and/or improve at least one agronomic trait of interest and/or improve plant health.

### II. Bacterial Strains

Various biocontrol agents or bacterial strains are provided which can be used to control one or more plant disease and/or improve at least one agronomic trait of interest and/or improve plant health. Such bacterial strains include AIP27511 (a Bacillus drentensis strain), AIP35174 (a Bacillus thuringiensis strain, AIP25773 (a Bacillus flexus strain), AIP15251 (a Bacillus frigoritolerans strain), AIP61892 (a Bacillus subtilus subsp. Subtilus strain), AIP79428 (a Burkholderia vietnamiensis strain), AIP14931 (a Bacillus thuringiensis strain), AIP39589 (a Bacillus acidiceler strain), and AIP36895 (a Bacillus simplex strain). Cell populations comprising one or more of AIP27511, AIP35174, AIP25773, AIP15251, AIP61892, AIP79428, AIP14931, AIP39589, and AIP36895 are provided, as well as, populations of spores derived from each of these strains, or any preparation thereof.

Thus, various bacterial strains and/or the pesticidal compositions provided herein comprise as an active ingredient (a) a cell population comprising one or more of AIP27511, AIP35174, AIP25773, AIP15251, AIP61892, AIP79428, AIP14931, AIP39589, and AIP36895, or an active variant of any thereof, or (b) a population of spores formed from one or more of AIP27511, AIP35174, AIP25773, AIP15251, AIP61892, AIP79428, AIP14931, AIP39589, and AIP36895, or an active variant of any thereof.

AIP27511 was deposited with the Patent Depository of the National Center for Agricultural Utilization Research Agricultural Research Service, U.S. Department of Agriculture, 1815 North University Street, Peoria, Ill. 61604 U.S.A. on Aug. 6, 2015 and assigned NRRL No. B-67082.

AIP35174 was deposited with the Patent Depository of the National Center for Agricultural Utilization Research Agricultural Research Service, U.S. Department of Agriculture, 1815 North University Street, Peoria, Ill. 61604 U.S.A. on Aug. 6, 2015 and assigned NRRL No. B-67084.

AIP25773 was deposited with the Patent Depository of the National Center for Agricultural Utilization Research Agricultural Research Service, U.S. Department of Agriculture, 1815 North University Street, Peoria, Ill. 61604 U.S.A. on Aug. 6, 2015 and assigned NRRL No. B-67085.

AIP15251 was deposited with the Patent Depository of the National Center for Agricultural Utilization Research Agricultural Research Service, U.S. Department of Agriculture, 1815 North University Street, Peoria, Ill. 61604 U.S.A. on Aug. 6, 2015 and assigned NRRL No. B-67083.

AIP61892 was deposited with the Patent Depository of the National Center for Agricultural Utilization Research Agricultural Research Service, U.S. Department of Agriculture, 1815 North University Street, Peoria, Ill. 61604 U.S.A. on Aug. 6, 2015 and assigned NRRL No. B-67089.

AIP79428 was deposited with the Patent Depository of the National Center for Agricultural Utilization Research Agricultural Research Service, U.S. Department of Agriculture, 1815 North University Street, Peoria, Ill. 61604 U.S.A. on Aug. 6, 2015 and assigned NRRL No. B-67081.

AIP14931 was deposited with the Patent Depository of the National Center for Agricultural Utilization Research Agricultural Research Service, U.S. Department of Agriculture, 1815 North University Street, Peoria, Ill. 61604 U.S.A. on Aug. 6, 2015 and assigned NRRL No. B-67088.

AIP39589 was deposited with the Patent Depository of the National Center for Agricultural Utilization Research Agricultural Research Service, U.S. Department of Agriculture, 1815 North University Street, Peoria, Ill. 61604 U.S.A. on Aug. 6, 2015 and assigned NRRL No. B-67087.

AIP36895 was deposited with the Patent Depository of the National Center for Agricultural Utilization Research Agricultural Research Service, U.S. Department of Agriculture, 1815 North University Street, Peoria, Ill. 61604 U.S.A. on Aug. 6, 2015 and assigned NRRL No. B-67086

Each of the deposits identified above will be maintained under the terms of the Budapest Treaty on the International Recognition of the Deposit of Microorganisms for the Purposes of Patent Procedure. Each deposit was made merely as a convenience for those of skill in the art and is not an admission that a deposit is required under 35 U.S.C. § 112.

The term “isolated” encompasses a bacterium, spore, or other entity or substance, that has been (1) separated from at least some of the components with which it was associated when initially produced (whether in nature or in an experimental setting), and/or (2) produced, prepared, purified, and/or manufactured by the hand of man. Isolated bacteria may be separated from at least about 10%, about 20%, about 30%, about 40%, about 50%, about 60%, about 70%, about 80%, about 90%, or more of the other components with which they were initially associated.

As used herein, a substance is “pure” if it is substantially free of other components. The terms “purify,” “purifying” and “purified” refer to a bacterium, spore, or other material that has been separated from at least some of the components with which it was associated either when initially produced or generated (e.g., whether in nature or in an experimental setting), or during any time after its initial production. A bacterium or spore or a bacterial population or a spore population may be considered purified if it is isolated at or after production, such as from a material or environment containing the bacterium or bacterial population or spore, and a purified bacterium or bacterial population or spore may contain other materials up to about 10%, about 20%, about 30%, about 40%, about 50%, about 60%, about 70%, about 80%, about 90%, or above about 90% and still be considered purified. In some embodiments, purified bacteria or spores and bacterial populations or spore populations are more than about 80%, about 85%, about 90%, about 91%, about 92%, about 93%, about 94%, about 95%, about 96%, about 97%, about 98%, about 99%, or more than about 99% pure. In specific embodiments, a culture of bacteria contains no other bacterial species in quantities to be detected by normal bacteriological techniques.

By “population” is intended a group or collection that comprises two or more (i.e., 10, 100, 1,000, 10,000, 1×10⁶, 1×10⁷, or 1×10⁸ or greater). Various compositions are provided herein that comprise a population of at least one bacterial strain. In specific embodiments, the population of at least one of a bacterial strain (i.e., AIP27511, AIP35174, AIP25773, AIP15251, AIP61892, AIP79428, AIP14931, AIP39589, and AIP36895, or an active variant of any thereof, or spores or forespores or a combination of cells, forespores and/or spores, formed from one or more of AIP27511, AIP35174, AIP25773, AIP15251, AIP61892, AIP79428, AIP14931, AIP39589, and AIP36895, or an active variant of any thereof) comprises a concentration of at least about 10⁵ CFU/ml to about 10¹¹ CFU/ml, about 10⁵ CFU/ml to about 10¹⁰ CFU/ml, about 10⁵ CFU/ml to about 10¹² CFU/ml, about 10⁵ CFU/ml to about 10⁶ CFU/ml, about 10⁶ CFU/ml to about 10⁷ CFU/ml, about 10⁷ CFU/ml to about 10⁸ CFU/ml, about 10⁸ CFU/ml to about 10⁹ CFU/ml, about 10⁹ CFU/ml to about 10¹⁰ CFU/ml, about 10¹⁰ CFU/ml to about 10¹¹ CFU/ml, about 10¹¹ CFU/ml to about 10¹² CFU/ml. In other embodiments, the concentration of the bacterial strain provided herein or active variant thereof comprises at least about 10⁵ CFU/ml, at least about 10⁶ CFU/ml, at least about 10⁷ CFU/ml, at least about 10⁸ CFU/ml, at least about 10⁹ CFU/ml, at least about 10¹⁰ CFU/ml, at least about 10¹¹ CFU/ml, or at least about 10¹² CFU/ml.

A “spore” refers to at least one dormant (at application) but viable reproductive unit of a bacteria species. Non-limiting methods by which spores are formed from each of AIP27511, AIP35174, AIP25773, AIP15251, AIP61892, AIP79428, AIP14931, AIP39589, and AIP36895 (or variants of any thereof) are disclosed elsewhere herein. It is further recognized the populations disclosed herein can comprise a combination of vegetative cells and forepores (cells in an intermediate stage of spore formation); a combination of forespores and spores; or a combination of forespores, vegetative cells and/or spores.

The compositions comprising a bacterial strain (i.e., at least one of AIP27511, AIP35174, AIP25773, AIP15251, AIP61892, AIP79428, AIP14931, AIP39589, and AIP36895, or an active variant of any thereof, or a spore or a forespore or a combination of cells, forespores or/and spores, from any one of AIP27511, AIP35174, AIP25773, AIP15251, AIP61892, AIP79428, AIP14931, AIP39589, or AIP36895, or an active variant of any thereof) can further comprise an agriculturally acceptable carrier. The term “agriculturally acceptable carrier” is intended to include any material that facilitates application of a composition to the intended subject (i.e, a plant or plant part susceptible to a plant disease of interest (i.e., Asian Soybean Rust (ASR), or any other disease discloses herein or a plant or plant part for improving an agronomic trait of interest). Carriers used in compositions for application to plants and plant parts are preferably non-phytotoxic or only mildly phytotoxic. A suitable carrier may be a solid, liquid or gas depending on the desired formulation. In one embodiment, carriers include polar or non-polar liquid carriers such as water, mineral oils and vegetable oils. Additional carriers are disclosed elsewhere herein.

A. Active Variants of a Bacterial Strain

Further provided are active variants of AIP27511, AIP35174, AIP25773, AIP15251, AIP61892, AIP79428, AIP14931, AIP39589, and AIP36895. Such variants will retain the ability to control one or more plant diseases (i.e., reduce disease severity and/or reduce disease development) and/or control one or more plant pathogens. In some embodiments, variants will retain the ability to control one or more fungal plant diseases and/or one or more fungal pathogens. In other embodiments, variants will retain the ability to control ASR.

Active variants of the various bacterial strains provided herein include, for example, any isolate or mutant of AIP27511, AIP35174, AIP25773, AIP15251, AIP61892, AIP79428, AIP14931, AIP39589, and AIP36895.

In specific embodiments, the bacterial strain is compatible with a biocide. A biocide is a chemical substance that can exert a controlling effect on an organism by chemical or biological means. Biocides include pesticides, such as fungicides; herbicides; insecticides, other crop protection chemicals, and the like. Such compounds are discussed in detail elsewhere herein. A bacterial strain is compatible with a biocide when the bacterial strain is able to survive and/or reproduce in the presence of an effective amount of a biocide of interest. In instances where the bacterial strain is not compatible for a biocide of interest, if desired, methods can be undertaken to modify the bacterial strain to impart the compatibility of interest. Such methods to produce modified bacterial strains include both selection techniques and/or transformation techniques.

By “modified bacterial strain” is intended a population wherein the strain has been modified (by selection and/or transformation) to have one or more additional traits of interest. In some cases the modified bacterial strain comprises any one of AIP27511, AIP35174, AIP25773, AIP15251, AIP61892, AIP79428, AIP14931, AIP39589, or AIP36895, or an active variant of any thereof. In specific embodiments, the modified bacterial strain is compatible with a biocide of interest, including but not limited to, resistance to a herbicide, fungicide, pesticide, or other crop protection chemical. The modified biocide-resistant strains have the same identification characteristics as the original sensitive strain except they are significantly more resistant to the particular herbicide, fungicide, pesticide, or other crop protection chemical. Their identification is readily possible by comparison with characteristics of the known sensitive strain. Thus, isolated populations of modified bacterial strains are provided.

An increase in resistance to a biocide (i.e., for example, a herbicide, fungicide, pesticide, or other crop protection chemical resistance) refers to the ability of an organism (i.e., bacterial cell or spore) to survive and reproduce following exposure to a dose of the biocide (e.g, herbicide, fungicide, pesticide, or other crop protection chemical) that would normally be lethal to the unmodified organism or would substantially reduce growth of the unmodified organism. In specific embodiments, the increase in resistance to a biocide is demonstrated in the presence of an agriculturally effective amount of the biocide.

In such instances, the modified bacterial strain having resistance to one or more biocides is useful for enhancing the competitiveness of bacterial strains particularly over other microbial agents which are not resistant to herbicides, fungicides, pesticides, or other crop protection chemicals. Therefore, compositions provided herein include selected or engineered bacterial strains and modified populations of bacterial strains. These bacterial strains or modified bacterial strains can be used as an inoculant for plants. They can also be applied as a spray application directly to the aerial parts of plants, and can be mixed with the herbicide or other chemical to which they have been modified to become tolerant.

Thus, active variants of the bacterial strains disclosed herein, include for example, a modified strain, such that the active variant controls a plant disease and further are able to grow in the presence of at least one biocide.

Recombinant bacterial strains having resistance to an herbicide, fungicide, pesticide, or other crop protection chemical can be made through genetic engineering techniques and such engineered or recombinant bacterial strains grown to produce a modified population of bacterial strains. A recombinant bacterial strain is produced by introducing polynucleotides into the bacterial host cell by transformation. Methods for transforming microorganisms are known and available in the art. See, generally, Hanahan, D. (1983) Studies on transformation of Escherichia coli with plasmids J. Mol. Biol. 166, 557-77; Seidman, C. E. (1994) In: Current Protocols in Molecular Biology, Ausubel, F. M. et al. eds., John Wiley and Sons, NY; Choi et al. (2006) J. Microbiol. Methods 64:391-397; Wang et al. 2010. J. Chem. Technol. Biotechnol. 85:775-778. Transformation may occur by natural uptake of naked DNA by competent cells from their environment in the laboratory. Alternatively, cells can be made competent by exposure to divalent cations under cold conditions, by electroporation, by exposure to polyethylene glycol, by treatment with fibrous nanoparticles, or other methods well known in the art.

Herbicide resistance genes for use in transforming a recombinant bacterial strain include, but are not limited to, fumonisin detoxification genes (U.S. Pat. No. 5,792,931); acetolactate synthase (ALS) mutants that lead to herbicide resistance, in particular the sulfonylurea-type herbicides, such as the S4 and/or Hra mutations; inhibitors of glutamine synthase such as phosphinothricin or basta (e.g., bar gene); and glyphosate resistance (EPSPS gene); gluphosinate, and HPPD resistance (WO 96/38576, U.S. Pat. Nos. 6,758,044; 7,250,561; 7,935,869; and 8,124,846), or other such genes known in the art. The disclosures of WO 96/38576, U.S. Pat. Nos. 5,792,931, 6,758,044; 7,250,561; 7,935,869; and 8,124,846 are herein incorporated by reference. The bar gene encodes resistance to the herbicide basta, the nptII gene encodes resistance to the antibiotics kanamycin and geneticin, and the ALS-gene mutants encode resistance to the sulfonylurea herbicides including chlorsulfuron, metsulfuron, sulfometuron, nicosulfuron, rimsulfuron, flazasulfuron, sulfosulfuron, and triasulfuron, and the imadizolinone herbicides including imazethapyr, imazaquin, imazapyr, and imazamethabenz.

To identify and produce a modified population of bacterial strains through selection, the bacterial strains are grown in the presence of the herbicide, fungicide, pesticide, or other crop protection chemical as the selection pressure. Susceptible agents are killed while resistant agents survive to reproduce without competition. As the bacterial strains are grown in the presence of the herbicide, fungicide, pesticide, or other crop protection chemical, resistant bacterial strains successfully reproduce and become dominant in the population, becoming a modified population of bacterial strains. Methods for selecting resistant strains are known and include U.S. Pat. Nos. 4,306,027 and 4,094,097, herein incorporated by reference. The active variant of the bacterial strain comprising a modified population of bacterial strains will have the same identification characteristics as the original sensitive strain except they are significantly more tolerant to the particular herbicide, fungicide, pesticide, or other crop protection chemical. Thus, their identification is readily possible by comparison with characteristics of the known sensitive strain.

Further active variants of the various bacteria provide herein can be identified employing, for example, methods that determine the sequence identity relatedness between the 16S ribosomal RNA, methods to identify groups of derived and functionally identical or nearly identical strains include Multi-locus sequence typing (MLST), concatenated shared genes trees, Whole Genome Alignment (WGA), Average Nucleotide Identity, and MinHash (Mash) distance metric.

In one aspect, the active variants of the bacterial strain(s) AIP27511, AIP35174, AIP25773, AIP15251, AIP61892, AIP79428, AIP14931, AIP39589, or AIP36895 include strains that are closely related to any of the disclosed strains by employing the Bishop MLST method of organism classification as defined in Bishop et al. (2009) BMC Biology 7(1)1741-7007-7-3. Thus, in specific embodiments, an active variant of a bacterial strain disclosed herein includes a bacterial strain that falls within at least a 80%, 85%, 86%, 87%, 88%, 89%, 90%, 91%, 92%, 93%. 94%, 95%, 96%, 97%, 98%, 98.5%, 98.8%, 99%, 99.1%, 99.2%, 99.3%, 99.4%, 99.5%, 99.6%, 99.7%, 99.8%, or 99.9% sequence cut off employing the Bishop method of organism classification as set forth in Bishop et al. (2009) BMC Biology 7(1)1741-7007-7-3, which is herein incorporated by reference in its entirety. Active variants of the bacteria identified by such methods will retain the ability to improve at least one agronomic trait when applied in an effective amount to a plant, plant part, or an area of cultivation, including for example, reducing plant disease severity and/or reducing plant disease development.

In another aspect, the active variant of the bacterial strain(s) disclosed herein include strains that are closely related to any of the disclosed strains on the basis of the Average Nucleotide Identity (ANI) method of organism classification. ANI (see, for example, Konstantinidis, K. T., et al., (2005) PNAS USA 102(7):2567-72; and Richter, M., et al., (2009) PNAS 106(45):19126-31) and variants (see, for example, Varghese, N. J., et al., Nucleic Acids Research (Jul. 6, 2015): gkv657) are based on summarizing the average nucleotides shared between the genomes of strains that align in WGAs. Thus, in specific embodiments, an active variant of bacterial strain AIP27511, AIP35174, AIP25773, AIP15251, AIP61892, AIP79428, AIP14931, AIP39589, or AIP36895 disclosed herein includes a bacterial stain that falls within at least a 90%, 95%, 96%, 97%, 97.5%, 98%, 98.5%, 98.8%, 99%, 99.5%, or 99.8% sequence cut off employing the ANI method of organism classification as set forth in Konstantinidis, K. T., et al., (2005) PNAS USA 102(7):2567-72, which is herein incorporated by reference in its entirety. Active variants of the bacteria identified by such methods will retain the ability to improve at least one agronomic trait when applied in an effective amount to a plant, plant part, or an area of cultivation, including for example, reducing plant disease severity and/or reducing plant disease development.

In another aspect, the active variants of the isolated bacterial strains) disclosed herein includes strain(s) that are closely related to any of the above strains (for example, closely related to AIP27511, AIP35174, AIP25773, AIP15251, AIP61892, AIP79428, AIP14931, AIP39589, or AIP36895) on the basis of 16S rDNA sequence identity. See Stackebrandt E, at al., “Report of the ad hoc committee for the re-evaluation of the species definition in bacteriology,” Int J Syst Evol Microbiol. 52(3): 1043-7 (2002) regarding use of 16S rDNA sequence identity for determining relatedness in bacteria. In an embodiment, the at least one strain is at least 95% identical to any of the above strains on the basis of 16S rDNA sequence identity, at least 96% identical to any of the above strains on the basis of 16S rDNA sequence identity, at least 97% identical to any of the above strains on the basis of 16S rDNA sequence identity, at least 98% to any of the above strains on the basis of 16S rDNA sequence identity, at least 98.5% identical to any of the above strains on the basis of 16S rDNA sequence identity, at least 99% identical to any of the above strains on the basis of 16S rDNA sequence identity, at least 99.5% to any of the above strains on the basis of 16S rDNA sequence identity or at least 100% to any of the above strains on the basis of 1.65 rDNA sequence identity. Active variants of the bacteria identified by such methods will retain the ability to improve at least one agronomic trait when applied in an effective amount to a plant, plant part, or an area of cultivation, including for example, reducing plant disease severity and/or reducing plant disease development.

The MinHash (Mash) distance metric is a comparison method that defines thresholds for hierarchical classification of microorganisms at high resolution and requires few parameters and steps (Ondov et al. (2016) Genome Biology 17:132). Mash distance strongly corresponds to Average Nucleotide Identity method (ANI) for hierarchical classification (See, Konstantinidis, K. T. et al. (2005) PNAS USA 102(7):2567-72, herein incorporated by reference in its entirety). That is, an ANI of 97% is approximately equal to a Mash distance of 0.03, such that values put forth as useful classification thresholds in the ANI literature can be directly applied with the Mash distance.

Active variants of the bacterial strain(s) disclosed herein include strains that are closely related to any of AIP27511, AIP35174, AIP25773, AIP15251, AIP61892, AIP79428, AIP14931, AIP39589, or AIP36895 on the basis of the Minhash (Mash) distance between complete genome DNA sequences. Thus, in specific embodiments, an active variant of a bacterial strain disclosed herein includes bacterial strains having a genome within a Mash distance of less than about 0.015 to the disclosed strains. In other embodiments, an active variant of a bacterial strain disclosed herein includes a distance metric of less than about 0.005, 0.010, 0.015, 0.020, 0.025, or 0.030. A genome, as it relates to the Mash distance includes both bacterial chromosomal DNA and bacterial plasmid DNA. In other embodiments, the active variant of a bacterial strain has a genome that is above a Mash distance threshold to the disclosed strains that is greater than dissimilarity caused by technical variance. In further instances, the active variant of a bacterial strain has a genome that is above a Mash distance threshold to the disclosed strains that is greater than dissimilarity caused by technical variance and has a Mash distance of less than about 0.015. In other instances, the active variant of a bacterial strain has a genome that is above a Mash distance threshold to the disclosed strains that is greater than dissimilarity caused by technical variance and has a Mash distance of less than about 0.005, 0.010, 0.015, 0.020, 0.025, or 0.030.

As used herein, “above technical variation” means above the Mash distance between two strains caused by errors in the genome assemblies provided the genomes being compared were each DNA sequenced with at least 20× coverage with the Illumina HiSeq 2500 DNA sequencing technology and the genomes are at least 99% complete with evidence for contamination of less than 2%. While 20× coverage is an art recognized term, for clarity, an example of 20× coverage is as follows: for a genome size of 5 megabases (MB), 100 MB of DNA sequencing from the given genome is required to have 20× sequencing coverage on average at each position along the genome. There are many suitable collections of marker genes to use for genome completeness calculations including the sets found in Campbell et al. (2013) PNAS USA 110(14):5540-45, Dupont et al. (2012) ISMEJ 6:1625-1628, and the CheckM framework (Parks et al. (2015) Genome Research 25:1043-1055); each of these references is herein incorporated in their entirety. Contamination is defined as the percentage of typically single copy marker genes that are found in multiple copies in the given genome sequence (e.g. Parks et al. (2015) Genome Research 25:1043-1055); each of these references is herein incorporated in their entirety. Completeness and contamination are calculated using the same collection of marker genes. Unless otherwise stated, the set of collection markers employed in the completeness and contamination assay is the set forth in Campbell et al. (2013) PNAS USA 110(14):5540-45, herein incorporated by reference.

Exemplary steps to obtain a distance estimate between the genomes in question are as follows: (1) Genomes of sufficient quality for comparison must be produced. A genome of sufficient quality is defined as a genome assembly created with enough DNA sequence to amount to at least 20× genome coverage using Illumina HiSeq 2500 technology. The genome must be at least 99% complete with contamination of less than 2% to be compared to the claimed microbe's genome. (2) Genomes are to be compared using the Minhash workflow as demonstrated in Ondov et al. (2016) Genome Biology 17:132, herein incorporated by reference in its entirety. Unless otherwise stated, parameters employed are as follows: “sketch” size of 1000, and “k-mer length” of 21. (3) Confirm that the Mash distance between the 2 genomes is less than 0.005, 0.010, 0.015, 0.020, 0.025, or 0.030. Active variants of the bacteria identified by such methods will retain the ability to improve at least one agronomic trait when applied in an effective amount to a plant, plant part, or an area of cultivation, including for example, reducing plant disease severity and/or reducing plant disease development.

### III. Formulations

The bacteria strains provided herein (i.e., AIP27511, AIP35174, AIP25773, AIP15251, AIP61892, AIP79428, AIP14931, AIP39589, or AIP36895, or active variant of any thereof, or a spore or a forespore or a combination of cells, forespores or/and spores, from any one of AIP27511, AIP35174, AIP25773, AIP15251, AIP61892, AIP79428, AIP14931, AIP39589, or AIP36895, or an active variant of any thereof) can be formulated as a cell paste, wettable powders, a cell pellet, dusts, granules, a slurry, a dry powder, aqueous or oil based liquid products, and the like. Such formulations will comprise the bacteria provided herein or an active variant thereof in addition to carriers and other agents. The formulations can be used in a variety of methods as disclosed elsewhere herein.

The bacterial strains disclosed herein and the active variants thereof can be formulated to include at least one or more of an extender, a solvent, spontaneity promoters, carriers, emulsifiers, dispersants, frost protectants, thickeners, and/or adjuvants.

Examples of typical formulations include water-soluble liquids (SL), emulsifiable concentrates (EC), emulsions in water (EW), suspension concentrates (SC, SE, FS, OD), water-dispersible granules (WG), granules (GR) and capsule concentrates (CS); WG; GR; BB; SG; ZC these and other possible types of formulation are described, for example, by Crop Life International and in Pesticide Specifications, Manual on development and use of FAO and WHO specifications for pesticides, FAO Plant Production and Protection Papers—173, prepared by the FAO/WHO Joint Meeting on Pesticide Specifications, 2004, ISBN: 9251048576. The formulations may comprise active agrochemical compounds other than one or more active compounds of the invention.

The formulations or application forms of the various bacterial strains or active variants thereof can comprise, but are not limited to, auxiliaries, such as extenders, solvents, spontaneity promoters, carriers, emulsifiers, dispersants, frost protectants, biocides, solid carriers, surfactants, thickeners and/or other auxiliaries, such as adjuvants. An adjuvant in this context is a component which enhances the biological effect of the formulation, without the component itself having a biological effect. Examples of adjuvants are agents which promote the retention, spreading, attachment to the leaf surface, or penetration.

Non-limiting extenders are, for example, water, polar and nonpolar organic chemical liquids, for example from the classes of the aromatic and non-aromatic hydrocarbons (such as paraffins, alkyl benzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide). If the extender used is water, it is also possible to employ, for example, organic solvents as auxiliary solvents. Essentially, non-limiting liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water. In principle it is possible to use any suitable solvent. Non-limiting solvents are, for example, aromatic hydrocarbons, such as xylene, toluene or alkylnaphthalenes, for example, chlorinated aromatic or aliphatic hydrocarbons, such as chlorobenzene, chloroethylene or methylene chloride, for example, aliphatic hydrocarbons, such as cyclohexane, for example, paraffins, petroleum fractions, mineral and vegetable oils, alcohols, such as methanol, ethanol, isopropanol, butanol or glycol, for example, and also their ethers and esters, ketones such as acetone, methyl ethyl ketone, methyl isobutyl ketone or cyclohexanone, for example, strongly polar solvents, such as dimethyl sulphoxide, and water.

Non-limiting examples of suitable carriers include, for example, ammonium salts and ground natural minerals such as kaolins, clays, talc, chalk, quartz, attapulgite, montmorillonite or diatomaceous earth, and ground synthetic minerals, such as finely divided silica, alumina and natural or synthetic silicates, resins, waxes and/or solid fertilizers. Mixtures of such carriers may likewise be used. Carriers suitable for granules include the following: for example, crushed and fractionated natural minerals such as calcite, marble, pumice, sepiolite, dolomite, and also synthetic granules of inorganic and organic meals, and also granules of organic material such as sawdust, paper, coconut shells, maize cobs, and tobacco stalks.

Liquefied gaseous extenders or solvents may also be used. Non-limiting examples are those extenders or carriers which at standard temperature and under standard pressure are gaseous, examples being aerosol propellants, such as halogenated hydrocarbons, and also butane, propane, nitrogen and carbon dioxide. Examples of emulsifiers and/or foam-formers, dispersants or wetting agents having ionic or nonionic properties, or mixtures of these surface-active substances, are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkylta urates), phosphoric esters of polyethoxylated alcohols or phenols, fatty acid esters of polyols, and derivatives of the compounds containing sulphates, sulphonates and phosphates, examples being alkylaryl polyglycol ethers, alkylsulphonates, alkyl sulphates, arylsulphonates, protein hydrolysates, lignin-sulphite waste liquors and methylcellulose. The presence of a surface-active substance is advantageous if one of the active compounds and/or one of the inert carriers is not soluble in water and if application takes place in water.

Further auxiliaries that may be present in the formulations and in the application forms derived from them include colorants such as inorganic pigments, examples being iron oxide, titanium oxide, Prussian Blue, and organic dyes, such as alizarin dyes, azo dyes and metal phthalocyanine dyes, and nutrients and trace nutrients, such as salts of iron, manganese, boron, copper, cobalt, molybdenum, and zinc.

Stabilizers, such as low-temperature stabilizers, preservatives, antioxidants, light stabilizers or other agents which improve chemical and/or physical stability may also be present. Additionally present may be foam-formers or defoamers.

Furthermore, the formulations and application forms derived from them may also comprise, as additional auxiliaries, stickers such as carboxymethylcellulose, natural and synthetic polymers in powder, granule or latex form, such as gum arabic, polyvinyl alcohol, polyvinyl acetate, and also natural phospholipids, such as cephalins and lecithins, and synthetic phospholipids. Further possible auxiliaries include mineral and vegetable oils.

There may possibly be further auxiliaries present in the formulations and the application forms derived from them. Examples of such additives include fragrances, protective colloids, binders, adhesives, thickeners, thixotropic substances, penetrants, retention promoters, stabilizers, sequestrants, complexing agents, humectants and spreaders. Generally speaking, the active compounds may be combined with any solid or liquid additive commonly used for formulation purposes.

Suitable retention promoters include all those substances which reduce the dynamic surface tension, such as dioctyl sulphosuccinate, or increase the viscoelasticity, such as hydroxypropylguar polymers, for example.

Suitable penetrants in the present context include all those substances which are typically used in order to enhance the penetration of active agrochemical compounds into plants. Penetrants in this context are defined in that, from the (generally aqueous) application liquor and/or from the spray coating, they are able to penetrate the cuticle of the plant and thereby increase the mobility of the active compounds in the cuticle. This property can be determined using the method described in the literature (Baur et al., 1997, Pesticide Science 51: 131-152). Examples include alcohol alkoxylates such as coconut fatty ethoxylate (10) or isotridecyl ethoxylate (12), fatty acid esters such as rapeseed or soybean oil methyl esters, fatty amine alkoxylates such as tallowamine ethoxylate (15), or ammonium and/or phosphonium salts such as ammonium sulphate or diammonium hydrogen phosphate, for example.

The various compositions and formulations disclosed herein can comprise an amount of a bacterial strain, such as AIP27511, AIP35174, AIP25773, AIP15251, AIP61892, AIP79428, AIP14931, AIP39589, or AIP36895, or active variant of any thereof, or a spore or a forespore or a combination of cells, forespores or/and spores, from any one of AIP27511, AIP35174, AIP25773, AIP15251, AIP61892, AIP79428, AIP14931, AIP39589, or AIP36895, or an active variant of any thereof. Such an amount can comprise a concentration of the bacterial strain of at least about 10⁴ to about 10¹¹, at least about 10⁵ CFU/gram to about 10¹¹ CFU/gram, about 10⁵ CFU/gram to about 10¹⁰ CFU/gram, about 10⁵ CFU/gram to about 10¹² CFU/gram, about 10⁵ CFU/gram to about 10⁶ CFU/gram, about 10⁶ CFU/gram to about 10⁷ CFU/gram, about 10⁷ CFU/gram to about 10⁸ CFU/gram, about 10⁸ CFU/gram to about 10⁹ CFU/gram, about 10⁹ CFU/gram to about 10¹⁰ CFU/gram, about 10¹⁰ CFU/gram to about 10¹¹ CFU/gram, or about 10¹¹ CFU/gram to about 10¹² CFU/gram. In other embodiments, the concentration of the bacterial strain comprises at least about 10⁵ CFU/gram, at least about 10⁶ CFU/gram, at least about 10⁷ CFU/gram, at least about 10⁸ CFU/gram, at least about 10⁹ CFU/gram, at least about 10¹⁰ CFU/gram, at least about 10¹¹ CFU/gram, at least about 10¹² CFU/gram, at least about 10⁴ CFU/gram. Such concentrations of the bacterial strain can occur in any formulation type of interest, including, for example in a wettable power, spray dried formulation, or in a cell paste.

Cell pastes and wettable powers and spray dried formulations can comprise a bacterial strain, such as AIP27511, AIP35174, AIP25773, AIP15251, AIP61892, AIP79428, AIP14931, AIP39589, or AIP36895, or active variant of any thereof, or a spore or a forespore or a combination of cells, forespores or/and spores, from any one of AIP27511, AIP35174, AIP25773, AIP15251, AIP61892, AIP79428, AIP14931, AIP39589, or AIP36895, or an active variant of any thereof. The amount of the bacterial strain can comprise a concentration of the bacterial strain of at least about 10⁵ CFU/gram to about 10¹¹ CFU/gram, about 10⁷ CFU/gram to about 10¹⁰ CFU/gram, about 10⁷ CFU/gram to about 10¹¹ CFU/gram, about 10⁶ CFU/gram to about 10¹⁰ CFU/gram, about 10⁶ CFU/gram to about 10¹¹ CFU/gram, about 10¹¹ CFU/gram to about 10¹² CFU/gram, about 10⁵ CFU/gram to about 10¹⁰ CFU/gram, about 10⁵ CFU/gram to about 10¹² CFU/gram, about 10⁵ CFU/gram to about 10⁶ CFU/gram, about 10⁶ CFU/gram to about 10⁷ CFU/gram, about 10⁷ CFU/gram to about 10⁸ CFU/gram, about 10⁸ CFU/gram to about 10⁹ CFU/gram, about 10⁹ CFU/gram to about 10¹⁰ CFU/gram, about 10¹⁰ CFU/gram to about 10¹¹ CFU/gram, or about 10¹¹ CFU/gram to about 10¹² CFU/gram. In some embodiments, the concentration of the bacterial strain comprises at least about 10⁵ CFU/gram, at least about 10⁶ CFU/gram, at least about 10⁷ CFU/gram, at least about 10⁸ CFU/gram, at least about 10⁹ CFU/gram, at least about 10¹⁰ CFU/gram, at least about 10¹¹ CFU/gram, at least about 10¹² CFU/gram, or at least about 10¹³ CFU/gram.

As used herein, a “cell paste” comprises a population of cells that has been centrifuged and/or filtered or otherwise concentrated.

Further provided is a coated seed which comprises a seed and a coating on the seed, wherein the coating comprises at least one bacterial strain, such as AIP27511, AIP35174, AIP25773, AIP15251, AIP61892, AIP79428, AIP14931, AIP39589, or AIP36895, or active variant of any thereof, or a spore or a forespore or a combination of cells, forespores or/and spores, from any one of AIP27511, AIP35174, AIP25773, AIP15251, AIP61892, AIP79428, AIP14931, AIP39589, or AIP36895, or an active variant of any thereof, wherein said bacterial strain or the active variant thereof is present on the seed at about 10⁵ CFU/seed to about 10⁷ CFU/seed, at about 10⁴ CFU/seed to about 10⁸ CFU/seed, at about 10⁴ CFU/seed to about 10⁵ CFU/seed, at about 10⁵ CFU/seed to about 10⁶ CFU/seed, at about 10⁶ CFU/seed to about 10⁷ CFU/seed, or at about 10⁷ CFU/seed to about 10⁸ CFU/seed. Various plants of interest are disclosed elsewhere herein.

A seed coating can further comprise at least at least one nutrient, at least one herbicide or at least one pesticide, or at least one biocide. See, for example, US App Pub. 20040336049, 20140173979, and 20150033811.

The various formulations disclosed herein can be stable for at least 30, 40, 50, 60, 70, 80, 90, 100, 125, 150, 200, 225, 250, 275, 300, 325, 350 days, 1.5 years, 2 years or longer. By stable is intened that the formulation retains viable bacteria and/or retains an effective amount of a biologically active bacteria. In one embodiment, the stable formulation retains at least about 1%, about 10%, about 20%, about 30% about 40%, about 50%, about 60%, about 70%, about 80%, or about 90% of CFU/gram in the formulation at a given storage time point when compared to the CFU/gram produced after immediate preparation of the formulation. In another embodiment, the stable formulation retains at least about 30% to 80%, about 50% to about 80%, about 60% to about 70%, about 70% to about 80%, about 40% to about 50%, about 50% to about 60%, about 60% to about 70% of biological activity in the formulation at a given storage time point when compared to the biological activity found in the formulation immediately after production. In another embodiment, the stable formulation at a given storage time point retains at least about 30%, 45%, 50%, 60%, 70%, 80%, 90% of biological activity when compared to the biological activity found in the formulation immediately after production. In still another embodiment, the stable formation retains any combination of the viability and biological activity noted above.

The formulations preferably comprise between 0.00000001% and 98% by weight of active compound or, with particular preference, between 0.01% and 95% by weight of active compound, more preferably between 0.5% and 90% by weight of active compound, based on the weight of the formulation.

The active compound content of the application forms prepared from the formulations may vary within wide ranges. The active compound concentration of the application forms may be situated typically between 0.00000001% and 95% by weight of active compound, preferably between 0.00001% and 1% by weight, based on the weight of the application form. Application takes place in a customary manner adapted to the application forms.

Moreover, the bacterial strain provided herein or an active variant thereof can be mixed with a biocide, such as a fungicide, insecticide, or herbicide to enhance its activity or the activity of the chemical to which it has been added. In some cases, the combination of the bacterial strain and chemical may show synergistic activity where the mixture of the two exceeds that expected from their simple additive effect.

In specific embodiments, the bacterial strain or active variant thereof is compatible with agricultural chemicals used to improve performance of biocides. Such agricultural chemicals include safeners, surfactants, stickers, spreaders, UV protectants, and suspension and dispersal aids. Safeners are chemicals that improve or modify the performance of herbicides. Surfactants, spreaders, and stickers are chemicals included in agricultural spray preparations that change the mechanical properties of the spray (for example, by altering surface tension or improving leaf cuticle penetration). UV protectants improve the performance of agricultural biocides by reducing degradation by ultraviolet light. Suspension and dispersal aids improve the performance of biocides by altering their behavior in a spray tank. In instances where the bacterial strain or active variant is not compatible with an agricultural chemical of interest, if desired, methods can be undertaken to modify the bacterial strain to impart the compatibility of interest. Such methods to produce modified bacterial strains include both selection techniques and/or transformation techniques.

The bacterial strain provided herein can be used to significantly improve at least one agronomic trait of interest (i.e, reduce disease such as ASR or another fungal pathogen of interest). The bacterial strain provided herein can be used with other pesticides for an effective integrated pest management program. In one embodiment, the biocontrol populations can be mixed with known pesticides in a manner described in WO 94/10845, herein incorporated by reference.

Non-limiting examples of compounds and compositions that can be added to the formulation, include but are not limited to, Acetyl tributyl citrate [Citric acid, 2-(acetyloxy)-, tributyl ester]; Agar; Almond hulls; Almond shells; alpha-Cyclodextrin; Aluminatesilicate; Aluminum magnesium silicate [Silicic acid, aluminum magnesium salt]; Aluminum potassium sodium silicate [Silicic acid, aluminum potassium sodium salt]; Aluminum silicate; Aluminum sodium silicate [Silicic acid, aluminum sodium salt]; Aluminum sodium silicate (1:1:1) [Silicic acid (H4SiO4), aluminum sodium salt (1:1:1)]; Ammonium benzoate [Benzoic acid, ammonium salt]; Ammonium stearate [Octadecanoic acid, ammonium salt]; Amylopectin, acid-hydrolyzed, 1-octenylbutanedioate; Amylopectin, hydrogen 1-octadecenylbutanedioate; Animal glue; Ascorbyl palmitate; Attapulgite-type clay; Beeswax; Bentonite; Bentonite, sodian; beta-Cyclodextrin; Bone meal; Bran; Bread crumbs; (+)-Butyl lactate; [Lactic acid, n-butyl ester, (S)]; Butyl lactate [Lactic acid, n-butyl ester]; Butyl stearate [Octadecanoic acid, butyl ester]; Calcareous shale; Calcite (Ca(Co₃)); Calcium acetate; Calcium acetate monohydrate [Acetic acid, calcium salt, monohydrate]; Calcium benzoate [Benzoic acid, calcium salt]; Calcium carbonate; Calcium citrate [Citric acid, calcium salt]; Calcium octanoate; Calcium oxide silicate (Ca₃O(SiO₄)); Calcium silicate [Silicic acid, calcium salt]; Calcium stearate [Octadecanoic acid, calcium salt]; Calcium sulfate; Calcium sulfate dehydrate; Calcium sulfate hemihydrate; Canary seed; Carbon; Carbon dioxide; Carboxymethyl cellulose [Cellulose, carboxymethyl ether]; Cardboard; Carnauba wax; Carob gum [Locust bean gum]; Carrageenan; Caseins; Castor oil; Castor oil, hydrogenated; Cat food; Cellulose; Cellulose acetate; Cellulose, mixture with cellulose carboxymethyl ether, sodium salt; Cellulose, pulp; Cellulose, regenerated; Cheese; Chlorophyll a; Chlorophyll b; Citrus meal; Citric acid; Citric acid, monohydrate; Citrus pectin; Citrus pulp; Clam shells; Cocoa; Cocoa shell flour; Cocoa shells; Cod-liver oil; Coffee grounds; Cookies; Cork; Corn cobs; Cotton; Cottonseed meal; Cracked wheat; Decanoic acid, monoester with 1,2,3-propanetriol; Dextrins; Diglyceryl monooleate [9-Octadecenoic acid, ester with 1,2,3-propanetriol]; Diglyceryl monostearate [9-Octadecanoic acid, monoester with xybis(propanediol)]; Dilaurin [Dodecanoic acid, diester with 1,2,3-propanetriol]

Dipalmitin [Hexadecanoic acid, diester with 1,2,3-propanetriol]; Dipotassium citrate [Citric acid, dipotassium salt]; Disodium citrate [Citric acid, disodium salt]; Disodium sulfate decahydrate; Diatomaceous earth (less than 1% crystalline silica); Dodecanoic acid, monoester with 1,2,3-propanetriol; Dolomite; Douglas fir bark; Egg shells; Eggs; (+)-Ethyl lactate [Lactic acid, ethyl ester, (S)]; Ethyl lactate [Lactic acid, ethyl ester]; Feldspar; Fish meal; Fish oil (not conforming to 40 CFR 180.950); Fuller's earth; Fumaric acid; gamma-Cyclodextrin; Gelatins; Gellan gum; Glue (as depolymd. animal collagen); Glycerin [1,2,3-Propanetriol]; Glycerol monooleate [9-Octadecenoic acid (Z)-, 2,3-dihydroxypropyl ester]; Glyceryl dicaprylate [Octanoic acid, diester with 1,2,3-propanetriol]; Glyceryl dimyristate [Tetradecanoic acid, diester with 1,2,3-propanetriol]; Glyceryl dioleate [9-Octadecenoic acid (9Z)-, diester with 1,2,3-propanetriol]; Glyceryl distearate; Glyceryl monomyristate [Tetradecanoic acid, monoester with 1,2,3-propanetriol]; Glyceryl monooctanoate [Octanoic acid, monoester with 1,2,3-propanetriol]; Glyceryl monooleate [9-Octadecenoic acid (9Z)-, monoester with 1,2,3-propanetriol]; Glyceryl monostearate [Octadecanoic acid, monoester with 1,2,3-propanetriol]; Glyceryl stearate [Octadecanoic acid, ester with 1,2,3-propanetriol]; Granite; Graphite; Guar gum; Gum Arabic; Gum tragacanth; Gypsum; Hematite (Fe₂O₃); Humic acid; Hydrogenated cottonseed oil; Hydrogenated rapeseed oil; Hydrogenated soybean oil; Hydroxyethyl cellulose [Cellulose, 2-hydroxyethyl ether]; Hydroxypropyl cellulose [Cellulose, 2-hydroxypropyl ether]; Hydroxypropyl methyl cellulose [Cellulose, 2-hydroxypropyl methyl ether]; Iron magnesium oxide (Fe₂MgO₄); Iron oxide (Fe₂O₃); Iron oxide (Fe₂O₃); Iron oxide (Fe₃O₄); Iron oxide (FeO); Isopropyl alcohol [2-Propanol]; Isopropyl myristate; Kaolin; Lactose; Lactose monohydrate; Lanolin; Latex rubber; Lauric acid; Lecithins; Licorice extract; Lime (chemical) dolomitic; Limestone; Linseed oil; Magnesium carbonate [Carbonic acid, magnesium salt (1:1); Magnesium benzoate; Magnesium oxide; Magnesium oxide silicate (Mg₃O(Si₂O₅)₂), monohydrate; Magnesium silicate; Magnesium silicate hydrate; Magnesium silicon oxide (Mg₂Si₃O₈); Magnesium stearate [Octadecanoic acid, magnesium salt]; Magnesium sulfate; Magnesium sulfate heptahydrate; Malic acid; Malt extract; Malt flavor; Maltodextrin; Methylcellulose [Cellulose, methyl ether]; Mica; Mica-group minerals; Milk; N/A Millet seed; Mineral oil (U.S.P.); 1-Monolaurin [Dodecanoic acid, 2,3-dihydroxypropyl ester]; 1-Monomyristin [Tetradecanoic acid, 2,3-dihydroxypropyl ester]; Monomyristin [Decanoic acid, diester with 1,2,3-propanetriol]; Monopalmitin [Hexadecanoic acid, monoester with 1,2,3-propanetriol]; Monopotassium citrate [Citric acid, monopotassium salt; Monosodium citrate [Citric acid, monosodium salt]; Montmorillonite; Myristic acid; Nepheline syenite; Nitrogen; Nutria meat; Nylon; Octanoic acid, potassium salt; Octanoic acid, sodium salt; Oils, almond; Oils, wheat; Oleic acid; Oyster shells; Palm oil; Palm oil, hydrogenated; Palmitic acid [Hexadecanoic acid]; Paraffin wax; Peanut butter; Peanut shells; Peanuts; Peat moss; Pectin; Perlite; Perlite, expanded; Plaster of paris; Polyethylene; Polyglyceryl oleate; Polyglyceryl stearate; Potassium acetate [Acetic acid, potassium salt]; Potassium aluminum silicate, anhydrous; Potassium benzoate [Benzoic acid, potassium salt]; Potassium bicarbonate [Carbonic acid, monopotassium salt]; Potassium chloride; Potassium citrate [Citric acid, potassium salt]; Potassium humate [Humic acids, potassium salts]; Potassium myristate [Tetradecanoic acid, potassium salt]; Potassium oleate [9-Octadecenoic acid (9Z)-, potassium salt; Potassium ricinoleate [9-Octadecenoic acid, 12-hydroxy-, monopotassium salt, (9Z,12R)-]; Potassium sorbate [Sorbic acid, potassium salt]; Potassium stearate [Octadecanoic acid, potassium salt]; Potassium sulfate; Potassium sulfate [Sulfuric acid, monopotassium salt]; 1,2-Propylene carbonate [1,3-Dioxolan-2-one, 4-methyl-]; Pumice; Red cabbage color (expressed from edible red cabbage heads via a pressing process using only acidified water); Red cedar chips; Red dog flour; Rubber; Sawdust; Shale; Silica, amorphous, fumed (crystalline free); Silica, amorphous, precipated and gel; Silica (crystalline free); Silica gel; Silica gel, precipitated, crystalline-free; Silica, hydrate; Silica, vitreous; Silicic acid (H₂SiO₃), magnesium salt (1:1);

Soap (The water soluble sodium or potassium salts of fatty acids produced by either the saponification of fats and oils, or the neutralization of fatty acid); Soapbark [Quillaja saponin]; Soapstone; Sodium acetate [Acetic acid, sodium salt]; Sodium alginate; Sodium benzoate [Benzoic acid, sodium salt]; Sodium bicarbonate; Sodium carboxymethyl cellulose [Cellulose, carboxymethyl ether, sodium salt]; Sodium chloride; Sodium citrate; Sodium humate [Humic acids, sodium salts]; Sodium oleate; Sodium ricinoleate [9-Octadecenoic acid, 12-hydroxy-, monosodium salt,

(9Z,12R)-]; Sodium stearate [Octadecanoic acid, sodium salt]; Sodium sulfate; Sorbitol [D-glucitol]; Soy protein; Soya lecithins [Lecithins, soya]; Soybean hulls; Soybean meal; Soybean, flour; Stearic acid [Octadecanoic acid]; Sulfur; Syrups, hydrolyzed starch, hydrogenated; Tetragylceryl monooleate [9-Octadecenoic acid (9Z)-, monoester with tetraglycerol]; Tricalcium citrate [Citric acid, calcium salt (2:3)]; Triethyl citrate [Citric acid, triethyl ester; Tripotassium citrate [Citric acid, tripotassium salt]; Tripotassium citrate monohydrate [Citric acid, tripotassium salt, monohydrate]; Trisodium citrate [Citric acid, trisodium salt]; Trisodium citrate dehydrate [Citric acid, trisodium salt, dehydrate]; Trisodium citrate pentahydrate [Citric acid, trisodium salt, pentahydrate]; Ultramarine blue [CI Pigment Blue 29]; Urea; Vanillia; Vermiculite; Vinegar (maximum 8% acetic acid in solution); Vitamin C [L-Ascorbic acid]; Vitamin; Walnut flour; Walnut shells; Wheat; Wheat flour; Wheat germ oil; Whey; White mineral oil (petroleum); Wintergreen oil; Wollastonite (Ca(SiO3)); Wool; Xanthan gum; Yeast; Zeolites (excluding erionite (CAS Reg. No. 66733-21-9)); Zeolites, NaA; Zinc iron oxide; Zinc oxide (ZnO); and Zinc stearate [Octadecanoic acid, zinc salt].

### IV. Methods of Use

The bacterial strains or modified bacterial strains or active variants thereof provided herein can be employed with any plant species to improve an agronomic trait of interest. Agonomic traits of interest include any trait that improves plant health or commercial value. Non-limiting examples of agronomic traits of interest including increase in biomass, increase in drought tolerance, thermal tolerance, herbicide tolerance, drought resistance, insect resistance, fungus resistance, virus resistance, bacteria resistance, male sterility, cold tolerance, salt tolerance, increased yield, enhanced nutrient use efficiency, increased nitrogen use efficiency, increased tolerance to nitrogen stress, increased fermentable carbohydrate content, reduced lignin content, increased antioxidant content, enhanced water use efficiency, increased vigor, increased germination efficiency, earlier or increased flowering, increased biomass, altered root-to-shoot biomass ratio, enhanced soil water retention, or a combination thereof. In other instance, the agronomic trait of interest includes an altered oil content, altered protein content, altered seed carbohydrate composition, altered seed oil composition, and altered seed protein composition, chemical tolerance, cold tolerance, delayed senescence, disease resistance, drought tolerance, ear weight, growth improvement, health enhancement, heat tolerance, herbicide tolerance, herbivore resistance, improved nitrogen fixation, improved nitrogen utilization, improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, or a detectable modulation in the proteome relative to a reference plant.

In one non-limiting embodiment, the bacterial strain or active variant thereof provided herein can be employed to decrease or reduce the level of a plant pest. “Pests” includes but is not limited to, insects, fungi, bacteria, nematodes, acarids, protozoan pathogens, animal-parasitic liver flukes, and the like. In one non-limiting embodiment, the bacterial strain or active variant thereof provided herein can be employed with any plant species susceptible to a plant disease. By “a plant susceptible to a plant disease” is meant that the causative pathogen(s) of the plant disease are able to infect the plant.

Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americans), fig (Ficus casica), guava (Psidium guajava), mango (Mangifera indica), olive (Olea europaea), papaya (Carica papaya), grape (Vitus spp.), strawberry (Fragaria x ananassa), cherry (Prunus spp.), apple (Malus domestica), orange (Citrus x sinensis) cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true first such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans, peas, and dry pulses. Beans include guar, locust bean, fenugreek, soybean, garden beans, cowpea, mungbean, lima bean, fava bean, lentils, chickpea, etc.

A. Non-Limiting Plant Pests

Examples of plant diseases which can be treated or reduced or prevented include, but are not limited to, plant diseases caused by fungi, viruses or viroids, bacteria, insects, nematodes, protozoa, and the like. Examples of fungal plant diseases include, but are not limited to, Asian Soybean Rust (ASR), gray mold, leaf spot, Frogeye Leaf Spot, Early Blight, Damping off complex, Brown Patch, black scurf, root rot, belly rot, sheath blight, Powdery Mildew, Anthracnose leaf spot, Downy Mildew, Pythium Blight, Late Blight, Fusarium Head Blight, sudden death syndrome (SDS), Fusarium Wilt, Corn Stalk Rot, Brown Rust, Black Rust, Yellow Rust, Wheat Rust, Rust, Apple Scab, Verticillium Wilt, Fire Blight, and Brown Rot, to name a few.

Plant pathogens of the invention include, but are not limited to, viruses or viroids, bacteria, insects, nematodes, fungi, and the like.

In specific embodiments, the bacterial strains provided herein are those that target one or more plant pathogens. For example, the various bacterial strains provided herein target one or more fungal pathogens that cause plant disease. For example, any of the bacterial strain provided herein or active variant thereof can have antifungal activity against one, two, three, four, five, or more fungal pathogens and/or fungal diseases described herein.

The methods and compositions disclosed herein can be used to control one or more fungal pathogens. A fungal pathogen can be, but is not limited to, a fungus selected from the group consisting of Botrytis spp., Botrytis cinerea, Cercospora spp, Cercospora sojina, Cercospora beticola, Alternaria spp., Alternaria solani, Rhizoctonia spp., Rhizoctonia solani, Blumeria graminis f. sp. Tritici, Erysiphe necator, Podosphaera xanthii, Golovinomyces cichoracearum, Erysiphe lagerstroemiae, Sphaerotheca pannosa, Colletotrichum cereale, Apiognomonia errabunda, Apiognomonia veneta, Colletotrichum spp, Colletotrichum gloeosporiodes, Discula fraxinea, Mycosphaerella spp., Phomopsis spp., Plasmopara viticola, Pseudoperonospora cubensis, Peronospora belbahrii, Bremia lactucae, Peronospora lamii, Plasmopara obduscens, Pythium spp., Pythium cryptoirregulare, Pythium aphanidermaturn, Pythium irregulare, Pythium sylvaticum, Pythium myriotylurn, Pythium ultimum, Phytophthora spp., Phytophthora capsici, Phytophthora nicotianae, Phytophthora infestans, Phytophthora tropicalis, Phytophthora sojae, Fusarium spp., Fusarium graminearum, Fusarium solani, Fusarium oxysporum, Fusarium graminicola, Gibberella zeae, Colletotrichum graminicola, Penicillium spp., Phakopsora sp., Phakopsora meibomiae, Phakopsora pachyrizi, Puccinia triticina, Puccinia recondita, Puccinia striiformis, Puccinia graminis, Puccinia spp., Sclerotium spp., Sclerotinia ssp., Venturia inaequalis, Verticillium spp, Erwinia amylovora, Monilinia spp., Monilinia fructicola, Monilinia lax, and Monilinia jructigena.

In some embodiments, the fungal pathogen is selected from the group consisting of Botrytis cinerea, Cercospora sojina, Alternaria solani, Rhizoctonia solani, Erysiphe necator, Podosphaera xanthii, Colletotrichum cereal, Plasmopara viticola, Peronospora belbahrii, Pythium aphanidermatum, Pythium sylvaticum, Pythium myriotylum, Pythium ultimum, Phytophthora nicotianae, Phytophthora infestans, Phytophthora tropicalis, Phytophthora sojae, Fusarium graminearum, Fusarium solani, Phakopsora pachyrizi, and Venturia inaequalis

In further embodiments, the fungal pathogen is Phakopsora sp., including Phakopsora pachyrhizi and/or Phakopsora meibomiae.

In specific embodiments, the bacterial strains provided herein are those that target one or more insect or insect pests. The term “insects” or “insect pests” as used herein refers to insects and other similar pests such as, for example, those of the order Acari including, but not limited to, mites and ticks. Insect pests of the present invention include, but are not limited to, insects of the order Lepidoptera, e.g. Achoroia grisella, Acleris gloverana, Acleris varlana, Adoxophyes orana, Agrotis ipsilon, Alabama argillacea, Alsophila pometaria, Amyelois transitella, Anagasta kuehniella, Anarsia lineatella, Anisota senatoria, Antheraea pernyi, Anticarsia gemmatalis, Archips sp., Argyrotaenia sp., Athetis mindara, Bombyx mori, Bucculatrix thurberiella, Cadra cautella, Choristoneura sp., Cochylls hospes, Colias eurytheme, Corcyra cephalonica, Cydia latiferreanus, Cydia pomonella, Datana integerrima, Dendrolimus sibericus, Desmiafeneralis, Diaphania hyalinata, Diaphania nitidalis, Diatraea grandiosella, Diatraea saccharalis, Ennomos subsignaria, Eoreuma loftini, Esphestia elutella, Erannis Warta, Estigmene acrea, Eulia salubricola, Eupocoellia ambiguella, Eupoecilia ambiguella, Euproctis chrysorrhoea, Euxoa messoria, Galleria mellonella, Grapholita molesta, Harrisina americana, Helicoverpa subflexa, Helicoverpa zea, Heliothis virescens, Hemileuca oliviae, Homoeosoma electellum, Hyphantia cunea, Keiferia lycopersicella, Lambdina fiscellaria fiscellaria, Lambdina fiscellaria lugubrosa, Leucoma salicis, Lobesia botrana, Loxostege sticticalis, Lymantria dispar, Macalla thyrisalis, Malacosoma sp., Mamestra brassicae, Mamestra configurata, Manduca quinquemaculata, Manduca sexta, Maruca testulalis, Melanchra pitta, Operophtera brumata, Orgyia sp., Ostrinia nubilalis, Paleacrita vernata, Papilio cresphontes, Pectinophora gossypiella, Phryganidia californica, Phyllonorycter blancardella, Pieris napi, Pieris rapae, Plathypena scabra, Platynota flouendana, Platynota stultana, Platyptilia carduidactyla, Plodia interpunctella, Plutella xylostella, Pontia protodice, Pseudaletia unipuncta, Pseudoplasia includens, Sabulodes aegrotata, Schizura concinna, Sitotroga cerealella, Spilonta ocellana, Spodoptera sp., Thaurnstopoea pityocampa, Tinsola bisselliella, Trichoplusia hi, Udea rubigalis, Xylomyges curiails, and Yponomeuta padella.

Insect pests also include insects selected from the orders Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, Coleoptera, etc.; particularly Lepidoptera. Insect pests of the invention for the major crops include, but are not limited to: Maize: Ostrinia nubilalis, European corn borer; Agrotis ipsilon, black cutworm; Helicoverpa zeae, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; western corn rootworm, e.g., Diabrotica virgifera virgifera; northern corn rootworm, e.g., Diabrotica longicornis barberi; southern corn rootworm, e.g., Diabrotica undecimpunctata howardi; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blotch leafminer; Anaphothrips obscrurus, grass thrips; Solenopsis milesta, thief ant; Tetranychus urticae, two spotted spider mite; Sorghum: Chilo partellus, sorghum borer; Spodoptera frugiperda, fall armyworm; Helicoverpa zea, corn earworm; Elasmopalpus lignosellus, leser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopaloszphum maidis; corn leaf aphid; Sipha flava, yellow sugarcane aphid; chinch bug, e.g., Blissus leucopterus leucopterus; Contarinia sorghicola, sorghum midge; Tetranychus cinnabarinus, carmine spider mite; Tetranychus urticae, two-spotted spider mite; Wheat: Pseudaletia umpunctata, army worm; Spodoptera frugiperda, fall armyworm; Elasmopalpus lignosellus, lesser cornstalk borer; Agrotis orthogonia, pale western cutworm; Elasmopalpus lignosellus, lesser cornstalk borer; Oulema melanopus, cereal leaf beetle; Hypera punctata, clover leaf weevil; southern corn rootworm, e.g., Diabrotica undecimpunctaka howardi; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguimpes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tuhpae, wheat curl mite; Sunflower: Cylindrocupturus adspersus, sunflower stem weevil; Smicronyx fulus, red sunflower seed weevil; Smicronyx sordidus, gray sunflower seed weevil; Suleima helianthana, sunflower bud moth; Homoeosoma electellum, sunflower moth; Zygogramma exclamationis, sunflower beetle; Bothyrus gibbosus, carrot beetle; Neolasioptera murtfeldtiana, sunflower seed midge; Cotton: Heliothis virescens, tobacco budworm; Helicoverpa zea, cotton bollworm; Spodoptera exigua, beet armyworm; Pectinophora gossypiella, pink bollworm; boll weevil, e.g., Anthonomus grandis; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentials, differential grasshopper; Thrips tabaci, onion thrips; Franklinkiella fusca, tobacco thrips; Tetranychus cinnabarinus, carmine spider mite; Tetranychus urticae, two-spotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhoper; chinch bug, e.g., Blissus leucopterus leucopterus; Acrosternum hilare, green stink bug; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Heliothis virescens, tobacco budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, two-spotted spider mite; Barley: Ostrinia nubilalis, European corn borer; Agrotis ipsilon, black cutworm; Schizaphis graminum, greenbug; chinch bug, e.g., Blissus leucopterus leucopterus; Acrosternum hilare, green stink bug; Euschistus servus, brown stink bug; Jylemya platura, seedcorn maggot; Mayetiola destructor, Hessian fly; Petrobia latens, brown wheat mite; Oil Seed Rape: Vrevicoryne brassicae, cabbage aphid; Phyllotreta cruciferae, crucifer flea beetle; Phyllotreta striolata, striped flea beetle; Phyllotreta nemorum, striped turnip flea beetle; Meligethes aeneus, rapeseed beetle; and the pollen beetles Meligethes rufimanus, Meligethes nigrescens, Meligethes canadianus, and Meligethes viridescens; Potato: Leptinotarsa decemlineata, Colorado potato beetle.

The methods and compositions provided herein can also be used against Hemiptera such as Lygus hesperus, Lygus lineolaris, Lygus pratensis, Lygus rugulipennis Popp, Lygus pabulinus, Calocoris norvegicus, Orthops compestris, Plesiocoris rugicollis, Cyrtopeltis modestus, Cyrtopeltis notatus, Spanagonicus albofasciatus, Diaphnocoris chlorinonis, Labopidicola allii, Pseudatomoscelis seriatus, Adelphocoris rapidus, Poecilocapsus lineatus, Blissus leucopterus, Nysius ericae, Nysius raphanus, Euschistus servus, Nezara viridula, Eurygaster, Coreidae, Pyrrhocoridae, Tinidae, Blostomatidae, Reduviidae, and Cimicidae. Pests of interest also include Araecerus fasciculatus, coffee bean weevil; Acanthoscelides obtectus, bean weevil; Bruchus rufmanus, broadbean weevil; Bruchus pisorum, pea weevil; Zabrotes subfasciatus, Mexican bean weevil; Diabrotica balteata, banded cucumber beetle; Cerotoma trifurcata, bean leaf beetle; Diabrotica virgifera, Mexican corn rootworm; Epitrix cucumeris, potato flea beetle; Chaetocnema confinis, sweet potato flea beetle; Hypera postica, alfalfa weevil; Anthonomus quadrigibbus, apple curculio; Sternechus paludatus, bean stalk weevil; Hypera brunnipennis, Egyptian alfalfa weevil; Sitophilus granaries, granary weevil; Craponius inaequahs, grape curculio; Sitophilus zeamais, maize weevil; Conotrachelus nenuphar, plum curculio; Euscepes postfaciatus, West Indian sweet potato weevil; Maladera castanea, Asiatic garden beetle; Rhizotrogus majalis, European chafer; Macrodactylus subspinosus, rose chafer; Tribohum confusum, confused flour beetle; Tenebrio obscurus, dark mealworm; Tribolium castaneum, red flour beetle; Tenebrio molitor, yellow mealworm.

Nematodes include parasitic nematodes such as root-knot, cyst, and lesion nematodes, including Heterodera spp., Meloidogyne spp., and Globodera spp.; particularly members of the cyst nematodes, including, but not limited to, Heterodera glycines (soybean cyst nematode); Heterodera schachtii (beet cyst nematode); Heterodera avenae (cereal cyst nematode); and Globodera rostochiensis and Globodera pailida (potato cyst nematodes). Lesion nematodes include Pratylenchus spp.

Insect pests can be tested for pesticidal activity of compositions of the invention in early developmental stages, e.g., as larvae or other immature forms. The insects may be reared in total darkness at from about 20 degree C. to about 30 degree C. and from about 30% to about 70% relative humidity. Bioassays may be performed as described in Czapla and Lang (1990) J. Econ. Entomol. 83 (6): 2480-2485. Methods of rearing insect larvae and performing bioassays are well known to one of ordinary skill in the art.

In further embodiments, the bacterial strains or active variants thereof (i.e., AIP27511, AIP35174, AIP25773, AIP15251, AIP61892, AIP79428, AIP14931, AIP39589, or AIP36895, or an active variant of any thereof, or a spore, or a forespore or a combination of cells, forespores and/or spores, from any one of AIP27511, AIP35174, AIP25773, AIP15251, AIP61892, AIP79428, AIP14931, AIP39589, or AIP36895, or an active variant of any thereof) control at least one, two, three, four, five, or more of the fungal diseases and/or fungal pathogens described herein.

In further embodiments, the bacterial strains or active variants thereof (i.e., AIP27511, AIP35174, AIP25773, AIP15251, AIP61892, AIP79428, AIP14931, AIP39589, or AIP36895, or an active variant of any thereof, or a spore, or a forespore or a combination of cells, forespores and/or spores, from any one of AIP27511, AIP35174, AIP25773, AIP15251, AIP61892, AIP79428, AIP14931, AIP39589, or AIP36895, or an active variant of any thereof) control at least one, two, three, four, five, or more fungal diseases selected from the group consisting of Asian Soybean Rust, gray mold, leaf spot, Frogeye Leaf Spot, Early Blight, Damping off complex, Brown Patch, black scurf, root rot, belly rot, sheath blight, Powdery Mildew, Anthracnose leaf spot, Downy Mildew, Pythium Blight, Late Blight, Fusarium Head Blight, SDS, Fusarium Wilt, Corn Stalk Rot, Brown Rust, Black Rust, Yellow Rust, Wheat Rust, Rust, Apple Scab, Verticillium Wilt, Fire Blight, and Brown Rot.

In further embodiments, the bacterial strain or active variant thereof (i.e., AIP27511, AIP35174, AIP25773, AIP15251, AIP61892, AIP79428, AIP14931, AIP39589, or AIP36895, or an active variant of any thereof, or a spore, or a forespore or a combination of cells, forespores and/or spores, from any one of AIP27511, AIP35174, AIP25773, AIP15251, AIP61892, AIP79428, AIP14931, AIP39589, or AIP36895, or an active variant of any thereof) control at least one, two, three, four, five, or more fungal diseases selected from the group consisting of Asian Soybean Rust, gray mold, Frogeye Leaf Spot, Early Blight, Damping off complex, Brown Patch, Powdery Mildew, Anthracnose leaf spot, Downy Mildew, Pythium Blight, Late Blight, Fusarium Head Blight, SDS, and Apple Scab.

In further embodiments, the bacterial strains or active variants thereof (i.e., AIP27511, AIP35174, AIP25773, AIP15251, AIP61892, AIP79428, AIP14931, AIP39589, or AIP36895, or an active variant of any thereof, or a spore, or a forespore or a combination of cells, forespores and/or spores, from any one of AIP27511, AIP35174, AIP25773, AIP15251, AIP61892, AIP79428, AIP14931, AIP39589, or AIP36895, or an active variant of any thereof) control at least one, two, three, four, five, or more fungal pathogens selected from the group consisting of Botrytis cinerea, Cersospora spp. Cercospora sojina, Cercospora beticola, Alternaria solani, Rhizoctonia solani, Blumeria graminis f. sp. Tritici, Erysiphe necator, Podosphaera xanthii, Golovinomyces cichoracearum, Erysiphe lagerstroemiae, Sphaerotheca pannosa, Colletotrichum cereale, Apiognomonia errabunda, Apiognomonia veneta, Colletotrichum gloeosporiodes, Discula fraxinea, Plasmopara viticola, Pseudoperonospora cubensis, Peronospora belbahrii, Bremia lactucae, Peronospora lamii, Plasmopara obduscens, Pythium cryptoirregulare, Pythium aphanidermatum, Pythium irregulare, Pythium sylvaticum, Pythium myriotylum, Pythium ultimum, Phytophthora capsici, Phytophthora nicotianae, Phytophthora infestans, Phytophthora tropicalis, Phytophthora sojae, Fusarium graminearum, Fusarium solani, Fusarium oxysporum, Fusarium graminicola, Gibberella zeae, Colletotrichum graminicola, Phakopsora sp., Phakopsora meibomiae, Phakopsora pachyrizi, Puccinia triticina, Puccinia recondita, Puccinia striiformis, Puccinia graminis, Puccinia spp., Venturia inaequalis, Verticillium spp Erwinia amylovora, Monihnia fructicola, Monilinia lax, andMonilinia fructigena.

In further embodiments, the bacterial strains or active variants thereof (i.e., AIP27511, AIP35174, AIP25773, AIP15251, AIP61892, AIP79428, AIP14931, AIP39589, or AIP36895, or an active variant of any thereof, or a spore, or a forespore or a combination of cells, forespores and/or spores, from any one of AIP27511, AIP35174, AIP25773, AIP15251, AIP61892, AIP79428, AIP14931, AIP39589, or AIP36895, or an active variant of any thereof) control at least one, two, three, four, five, or more fungal pathogens selected from the group consisting of Botrytis cinerea, Cercospora sojina, Alternaria solani, Rhizoctonia solani, Erysiphe necator, Podosphaera xanthii, Colletotrichum cereal, Plasmopara viticola, Peronospora belbahrii, Pythium aphanidermaturn, Pythium sylvaticum, Pythium myriotylum, Pythium ultimum, Phytophthora nicotianae, Phytophthora infestans, Phytophthora tropicalis, Phytophthora sojae, Fusarium graminearum, Fusarium solani, Phakopsora pachyrizi, and Venturia inaequalisa.

In further embodiments, the bacterial strains or active variants thereof (i.e., AIP27511, AIP35174, AIP25773, AIP15251, AIP61892, AIP79428, AIP14931, AIP39589, or AIP36895, or an active variant of any thereof, or a spore, or a forespore or a combination of cells, forespores and/or spores, from any one of AIP27511, AIP35174, AIP25773, AIP15251, AIP61892, AIP79428, AIP14931, AIP39589, or AIP36895, or an active variant of any thereof) control at least one, two, or all of Phakopsora. In further embodiments, the bacterial strain or modified biological agents disclosed herein (i.e., AIP27511, AIP35174, AIP25773, AIP15251, AIP61892, AIP79428, AIP14931, AIP39589, or AIP36895, or an active variant of any thereof, or a spore, or a forespore or a combination of cells, forespores and/or spores from any one of AIP27511, AIP35174, AIP25773, AIP15251, AIP61892, AIP79428, AIP14931, AIP39589, or AIP36895, or an active variant of any thereof) control at least one, or all of Phakopsora pachyrhizi and/or Phakopsora meibomiae. In other methods, the bacterial strains or modified bacterial strains disclosed herein (i.e., AIP27511, AIP35174, AIP25773, AIP15251, AIP61892, AIP79428, AIP14931, AIP39589, or AIP36895, or an active variant of any thereof, or a spore, or a forespore or a combination of cells, forespores and/or spores from any one of AIP27511, AIP35174, AIP25773, AIP15251, AIP61892, AIP79428, AIP14931, AIP39589, or AIP36895, or an active variant of any thereof) control Phakopsora pachyrhizi.

B. Methods of Treating or Preventing Plant Disease

Provided herein are methods of treating or preventing a plant disease comprising applying to a plant having a plant disease or at risk of developing a plant disease an effective amount of at least one bacterial strain provided herein or an active variant thereof wherein the bacterial strain controls a plant pathogen that causes the plant disease. In certain embodiments, the bacterial strain provided herein or active variant thereof may comprise at least one of AIP27511, AIP35174, AIP25773, AIP15251, AIP61892, AIP79428, AIP14931, AIP39589, or AIP36895, or an active variant of any thereof; or a spore, or a forespore or a combination of cells, forespores and/or spores from any one of AIP27511, AIP35174, AIP25773, AIP15251, AIP61892, AIP79428, AIP14931, AIP39589, or AIP36895, or an active variant or any thereof. In some embodiments, the effective amount of the bacterial strain or active variant thereof comprises at least about 10¹² to 10¹⁶ CFU per hectare or least about 10⁴ to 10¹⁶ CFU per hectare, or least about 10⁵ to 10¹¹ CFU per hectare.

In some methods, the bacterial strain provided herein or an active variant thereof is an antipathogenic agent that treats or prevents one, two, three, four, five or more plant diseases. In other methods, the bacterial strain provided herein or an active variant thereof is an antifungal agent that treats or prevents one, two, three, four, five or more fungal plant diseases. The bacterial strain provided herein or an active variant thereof can be employed with any plant species susceptible to a plant disease of interest.

Examples of diseases causes by the fungal pathogens described herein are provided in Table 1. Also provided are non-limiting exemplary crop species that are susceptible to the plant diseases caused by the pathogens. For example, Table 1 shows that Bortrytis cinerea causes gray mold on all flowering crops. Therefore, a bacterial strain provided herein or active variant thereof that controls Bortrytis cinerea can be applied to a plant having gray mold or at risk of developing gray mold in order to treat or prevent gray mold in the plant. Similarly, Table 1 shows that Rhizoctonia solani causes Damping off complex in corn, Damping off complex in soybean, Brown Patch in turf, and Damping off complex in ornamentals. Therefore, a bacterial strain provided herein or active variant thereof that controls Rhizoctonia solani can be applied to a plant having Damping off complex and/or brown patch or at risk of developing Damping off complex and/or brown patch in order to treat or prevent Damping off complex and/or brown patch in the plant. In yet another example, Table 1 shows that Colletotrichum cereal, Apiognomonia errabunda, Apiognomonia veneta, Colletotrichum gloeosporiodes, Discula fraxinea cause Anthracnose leaf spot. Therefore, a bacterial strain provided herein or active variant thereof that controls one or more of Colletotrichum cereal, Apiognomonia errabunda, Apiognomonia veneta, Colletotrichum gloeosporiodes, Discula fraxinea can be applied to a plant having Anthracnose leaf spot or at risk of developing Anthracnose leaf spot in order to treat or prevent Anthracnose leaf spot in the plant.

TABLE 1

| Causal Pathogen | Disease | Crop-species |
|---|---|---|
| Botrytis cinerea | gray mold | all flowering crops |
| Cersospora spp | Leaf Spot | Ornamentals |
| Cercospora sojina | Frogeye leaf spot | Soybeans |
| Cercospora beticola | | beets, spinach, chard |
| Alternaria solani | Early Blight | solanaceous plants |
| Rhizoctonia solani | Damping off complex | Corn |
| Rhizoctonia solani | Damping off complex | Soybean |
| Rhizoctonia solani | Brown Patch | Turf |
| Rhizoctonia solani | Damping off complex | Ornamentals |
| Rhizoctonia solani | black scurf | potato |
| Rhizoctonia solani | root rot | sugar beet |
| Rhizoctonia solani | belly rot | cucurbit |
| Rhizoctonia solani | sheath blight | rice |
| Blumeria graminis f. sp. Tritici | Powdery Mildew | Wheat |
| Erysiphe necator | Powdery Mildew | Grape |
| Podosphaera xanthii | Powdery Mildew | Cucurbit |
| Golovinomyces cichoracearum | Powdery Mildew | Ornamentals |
| Erysiphe lagerstroemiae | Powdery Mildew | Ornamentals |
| Sphaerotheca pannosa | Powdery Mildew | Ornamentals |
| Colletotrichum cereale | Anthracnose leaf spot | Turf/grasses/cereal |
| Apiognomonia errabunda | Anthracnose leaf spot | Turf/grasses/cereal |
| Apiognomonia veneta | Anthracnose leaf spot | Turf/grasses/cereal |
| Colletotrichum gloeosporiodes | Anthracnose leaf spot | Turf/grasses/cereal |
| Discula fraxinea | Anthracnose leaf spot | Turf/grasses/cereal |
| Plasmopara viticola | Downy Mildew | Grape |
| Pseudoperonospora cubensis | Downy Mildew | Cucurbit |
| Peronospora belbahrii | Downy Mildew | Basil |
| Bremia lactucae | Downy Mildew | Lettuce |
| Peronospora lamii | Downy Mildew | Coleus |
| Plasmopara obduscens | Downy Mildew | Impatiens |
| Pythium cryptoirregulare | Damping off complex | Ornamental Plants |
| Pythium aphanidermatum | Pythium Blight/Damping off complex | turf/ornamentals/row crop |
| Pythium irregulare | Damping off complex | turf/ornamentals/row crop |
| Pythium sylvaticum | Damping off complex | turf/ornamentals/row crop |
| Pythium myriotylum | Damping off complex | turf/ornamentals/row crop |
| Pythium ultimum | Pythium Blight/Damping off complex | turf/ornamentals/row crop |
| Phytophthora capsici | | cucurbit/pepper |
| Phytophthora nicotianae | | ornamental plants |
| Phytophthora infestans | Late Blight | solanaceous plant |
| Phytophthora tropicalis | | ornamental plants/peppers/ |
| | | tropical nut trees |
| Phytophthora sojae | | Soybean |
| Fusarium graminearum | Fusarium Head Blight | Cereals-Wheat |
| Fusarium solani | SDS | Soybean |
| Fusarium oxysporum | Fusarium Wilt | Herbaceous Plants |
| Fusarium graminicola | Corn Stalk Rot | Maize |
| Gibberella zeae | Corn Stalk Rot | Maize |
| Colletotrichum graminicola | Corn Stalk Rot | Maize |
| Phakopsora pachyrizi | Asian Soybean Rust | Soybean |
| Puccinia triticina | Brown Rust | Cereals |
| Puccinia recondita | Black Rust | Cereals |
| Puccinia striiformis | Yellow Rust | Cereals |
| Puccinia graminis | Wheat Rust | Cereals |
| Puccinia spp. | Rust | Ornamentals |
| Venturia inaequalis | Apple Scab | Malus |
| Verticillium spp | Verticillium Wilt | All |
| Erwinia amylovora | Fire Blight | Rosacea family |
| Monilinia fructicola | Brown Rot | Stone Fruits |
| Monilinia laxa | Brown Rot | Stone Fruits |
| Monilinia fructigena | Brown Rot | Stone Fruits |

Also provided herein are methods of treating or preventing Asian Soybean Rust (ASR) comprising applying to a plant having ASR or at risk of developing ASR an effective amount of at least one bacterial strain provided herein or an active variant thereof comprising AIP27511, AIP35174, AIP25773, AIP15251, AIP61892, AIP79428, AIP14931, AIP39589, or AIP36895, or an active variant of any thereof; or a spore, or a forespore or a combination of cells, forespores and/or spores from any one of AIP27511, AIP35174, AIP25773, AIP15251, AIP61892, AIP79428, AIP14931, AIP39589, or AIP36895, or an active variant of any thereof. In certain embodiments, the effective amount of the bacterial strain provided herein or an active variant thereof comprises at least about 10¹² to 10¹⁶ CFU per hectare and wherein the bacterial strain provided herein or active variant thereof controls a plant pathogen that causes ASR. In one embodiment, an effective amount of at least one bacterial strain provided herein or active variant thereof provided herein is used as a foliar application on a plant to treat or prevent ASR.

The bacterial strain provided herein or an active variant thereof or modified bacterial strain provided herein can be employed with any plant species susceptible to ASR. By “a plant susceptible to Asian Soybean Rust (ASR)” is meant that the causative pathogen(s) of ASR are able to infect the plant. Examples of plant species susceptible to ASR include, but are not limited to, soybean (Glycine max), common bean (Phaseolus vulgaris), such as green beans and kidney beans, lima beans (Phaseolus limensis), butter beans (Phaseolus lunatus), cowpeas (Vigna unguiculata), pigeon peas (Cajanus cajan), yam beans such as jicama (Pachyrhizus erosus). In a specific embodiment, a soybean plant is employed.

As outlined in further detail herein, in specific embodiments, the bacterial strain provided herein or an active variant thereof controls one or more fungi that causes ASR (such as, for example, Phakopsora). ASR is caused by one or more fungal pathogens of the genus Phakopsora. In non-limiting embodiments, the fungal pathogens that cause ASR are Phakopsora pachyrhizi or Phakopsora meibomiae. The ASR pathogen is well adapted for long-distance dispersal, because the spores can be readily carried by the wind, making it an ideal means for introduction to new, rust-free regions. The primary means of dissemination are spores, which can be carried by wind or splashed rain. These pathogens are obligate pathogens surviving and reproducing only on live hosts. In cultivated soybean, the first symptoms are light-brown polygonal lesions of 2 to 5 mm on the adaxial leaf surface. These lesions develop into volcano-shaped lesions known as pustules that appear on the abaxial surface of the leaf, where uredospores are produced.

In further embodiments, the bacterial strain provided herein or an active variant thereof controls Phakopsora pachyrhizi. In yet further embodiments, the bacterial strain provided herein or active variant thereof controls Phakopsora meibomiae. Various assays to measure such activity are disclosed elsewhere herein.

The term “treat” or “treating” or its derivatives includes substantially inhibiting, slowing, or reversing the progression of a condition, substantially ameliorating symptoms of a condition or substantially preventing the appearance of symptoms or conditions brought about by the pathogen that causes the plant disease.

The terms “controlling” and “protecting a plant from a pathogen” refers to one or more of inhibiting or reducing the growth, germination, reproduction, and/or proliferation of a pathogen of interest; and/or killing, removing, destroying, or otherwise diminishing the occurrence, and/or activity of a pathogen of interest. As such, a plant treated with the bacterial strain provided herein may show a reduced disease severity or reduced disease development in the presence of plant pathogens by a statistically significant amount.

The term “prevent” and is variations means the countering in advance of bacterial, fungal, viral, insect or other pest growth, proliferation, infestation, spore germination, and hyphae growth. In this instance, the composition is applied before exposure to the pathogens.

The term “ameliorate” and “amelioration” relate to the improvement in the treated plant condition brought about by the compositions and methods provided herein. The improvement can be manifested in the forms of a decrease in pathogen growth and/or an improvement in the diseased plant height, weight, number of leaves, root system, or yield. In general, the term refers to the improvement in a diseased plant physiological state.

The term “inhibit” and all variations of this term is intended to encompass the restriction or prohibition of bacterial, fungal, viral, nematode, insect, or any other pest growth, as well as spore germination.

The term “eliminate” relates to the substantial eradication or removal of bacteria, fungi, viruses, nematodes, insects, or any other pests by contacting them with the composition of the invention, optionally, according to the methods of the invention described below.

The terms “delay”, “retard” and all variations thereof are intended to encompass the slowing of the progress of bacterial, fungal, viral, nematode, insect, or any other pest growth, and spore germination. The expression “delaying the onset” is interpreted as preventing or slowing the progression of bacterial, fungal, viral, nematodes, insect, or any other pest growth, infestation, infection, spore germination and hyphae growth for a period of time, such that said bacterial, fungal, viral, nematode, insect, or any other pest growth, infestation, infection, spore germination and hyphae growth do not progress as far along in development, or appear later than in the absence of the treatment according to the invention.

A plant, plant part, or area of cultivation treated with the bacterial strain provided herein or an active variant thereof may show a reduced disease severity or reduced disease development in the presence of plant pathogens by a statistically significant amount. A reduced disease severity or reduced disease development can be a reduction of about 10% to about 20%, about 20% to about 30%, about 30% to about 40%, about 40% to about 50%, about 50% to about 60%, about 60% to about 70%, about 70% to about 80%, about 80% to about 90%, or about 90% to about 100% when compared to non-treated control plants. In other instances, the plant treated with a bacterial strain provided herein or an active variant thereof may show a reduced disease severity or reduced disease development in the presence of plant pathogen at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100% greater when compared to non-treated control plants. Methods for assessing plant disease severity are known, and include, measuring percentage of diseased leaf area (Godoy et al. (2006) Fitopatol. Bras. 31(1) 63-68 or by measuring uredinia counts (see Example 1).

By “antipathogenic compositions” or “antipathogenic” is intended that the compositions are capable of suppressing, controlling, preventing and/or killing the invading pathogenic organism. In specific embodiments, an antipathogenic composition reduces the disease symptoms resulting from pathogen challenge by a statistically significant amount, including for example, at least about 10% to at least about 20%, at least about 20% to about 50%, at least about 10% to about 60%, at least about 30% to about 70%, at least about 40% to about 80%, or at least about 50% to about 90% or greater. Hence, the methods of the invention can be utilized to protect plants from disease, particularly those diseases that are caused by plant pathogens.

Assays that measure antipathogenic activity are commonly known in the art, as are methods to quantitate disease resistance in plants following pathogen infection. See, for example, U.S. Pat. No. 5,614,395, herein incorporated by reference. Such techniques include, measuring over time, the average lesion diameter, the pathogen biomass, and the overall percentage of decayed plant tissues. For example, a plant either expressing an antipathogenic polypeptide or having an antipathogenic composition applied to its surface shows a decrease in tissue necrosis (i.e., lesion diameter) or a decrease in plant death following pathogen challenge when compared to a control plant that was not exposed to the antipathogenic composition. Alternatively, antipathogenic activity can be measured by a decrease in pathogen biomass. For example, a plant expressing an antipathogenic polypeptide or exposed to an antipathogenic composition is challenged with a pathogen of interest. Over time, tissue samples from the pathogen-inoculated tissues are obtained and RNA is extracted. The percent of a specific pathogen RNA transcript relative to the level of a plant specific transcript allows the level of pathogen biomass to be determined. See, for example, Thomma et al. (1998) Plant Biology 95:15107-15111, herein incorporated by reference.

Furthermore, in vitro antipathogenic assays include, for example, the addition of varying concentrations of the antipathogenic composition to paper disks and placing the disks on agar containing a suspension of the pathogen of interest. Following incubation, clear inhibition zones develop around the discs that contain an effective concentration of the antipathogenic polypeptide (Liu et al. (1994) Plant Biology 91:1888-1892, herein incorporated by reference). Additionally, microspectrophotometrical analysis can be used to measure the in vitro antipathogenic properties of a composition (Hu et al. (1997) Plant Mol. Biol. 34:949-959 and Cammue et al. (1992) J. Biol. Chem. 267: 2228-2233, both of which are herein incorporated by reference).

C. Methods of Inducing Disease Resistance in Plants and/or for Improving Plant Health and/or Improving an Agonomic Trait of Interest

Compositions and methods for inducing disease resistance in a plant to plant pathogens are also provided. Accordingly, the compositions and methods are also useful in protecting plants against fungal pathogens, viruses, nematodes, and insects. Provided herein are methods of inducing disease resistance against a plant pathogen comprising applying to a plant that is susceptible to a plant disease caused by the plant pathogen an effective amount of at least one bacterial strain provided herein or active variant thereof. In certain embodiments, the bacterial strain provided herein or active variant thereof may comprise at least one of AIP27511, AIP35174, AIP25773, AIP15251, AIP61892, AIP79428, AIP14931, AIP39589, or AIP36895, or an active variant of any thereof or a spore, or a forespore or a combination of cells, forespores and/or spores from any one of AIP27511, AIP35174, AIP25773, AIP15251, AIP61892, AIP79428, AIP14931, AIP39589, or AIP36895, or an active variant of any thereof. In certain embodiments, the bacterial strain provided herein or active variant thereof promotes a defensive response to the pathogen that causes the plant disease. In some embodiments, the effective amount of the bacterial strain provided herein or active variant thereof comprises at least about 10¹² to 10¹⁶ CFU per hectare.

A defensive response in the plant can be triggered after applying the bacterial strain provided herein or active variant thereof to the plant, but prior to pathogen challenge and/or after pathogen challenge of the plant treated with the bacterial strain provided herein or active variant thereof.

In some methods, the bacterial strain provided herein or active variant thereof induces resistance to one, two, three, four, five or more plant pathogens described herein. In other methods, the bacterial strain provided herein or active variant thereof induces resistance to one, two, three, four, five or more fungal plant pathogens described herein.

By “disease resistance” is intended that the plants avoid the disease symptoms that result from plant-pathogen interactions. That is, pathogens are prevented from causing plant diseases and the associated disease symptoms, or alternatively, the disease symptoms caused by the pathogen are minimized or lessened as compared to a control. Further provided are methods of improving plant health and/or improving an agronomic trait of interest comprising applying to a plant an effective amount of at least one bacterial strain provided herein or an active variant thereof or an active derivative thereof. In certain embodiments, the bacterial strain provided herein or active variant thereof may comprise at least one of AIP27511, AIP35174, AIP25773, AIP15251, AIP61892, AIP79428, AIP14931, AIP39589, or AIP36895, or an active variant of any thereof; or a spore, or a forespore or a combination of cells, forespores and/or spores from any one of AIP27511, AIP35174, AIP25773, AIP15251, AIP61892, AIP79428, AIP14931, AIP39589, or AIP36895, or an active variant of any thereof. In some embodiments, the effective amount of the bacterial strain provided herein or active variant thereof comprises at least about 10¹² to 10¹⁶ CFU per hectare.

By “improved plant health” is meant increased growth and/or yield of a plant, increased stress tolerance and/or decreased herbicide resistance, to name a few. Increased stress tolerance refers to an increase in the ability of a plant to decrease or prevent symptoms associated with one or more stresses. The stress can be a biotic stress that occurs as a result of damage done to plants by other living organisms such as a pathogen (for example, bacteria, viruses, fungi, parasites), insects, nematodes, weeds, cultivated or native plants. The stress can also be an abiotic stress such as extreme temperatures (high or low), high winds, drought, salinity, chemical toxicity, oxidative stress, flood, tornadoes, wildfires, radiation and exposure to heavy metals. Non-limiting examples of improved agronomic traits are disclosed elsewhere herein. In specific embodiments, an effective amount of the bacterial strain or active variant thereof improves plant health or improves an agronomic trait of interest by a statistically significant amount, including for example, at least about 10% to at least about 20%, at least about 20% to about 50%, at least about 10% to about 60%, at least about 30% to about 70%, at least about 40% to about 80%, or at least about 50% to about 90% or greater.

D. Methods of Application to a Plant or Plant Part

The bacterial strain provided herein or active variant thereof are applied in an effective amount. An effective amount of a bacterial strain provided herein or active variant thereof is an amount sufficient to control, treat, prevent, inhibit the pathogen that causes a plant disease, and/or reduce plant disease severity or reduce plant disease development. In other embodiments, the effective amount of the bacterial strain provided herein or active variant thereof is an amount sufficient to improve an agronomic trait of interest and/or to promote or increase plant health, growth or yield of a plant susceptible to a disease. The rate of application of the bacterial strain provided herein or active variant thereof may vary according to the pathogen being targeted, the crop to be protected, the efficacy of the bacterial strain provided herein or active variant thereof, the severity of the disease, the climate conditions, the agronomic trait of interest to improve, and the like.

Generally, the rate of bacterial strain provided herein or active variant thereof is 10⁷ to 10¹⁶ colony forming units (CFU) per hectare. In other embodiments, for a field inoculation, the rate of bacterial strain provided herein or active variant thereof application is 3×10⁷ to 1×10¹¹ colony forming units (CFU) per hectare. (This corresponds to about 1 Kg to 10 kg of formulated material per hectare). In other embodiments, for a field inoculation, the rate of bacterial strain provided herein or active variant thereof application is 3×10⁷ to 1×10¹⁶ colony forming units (CFU) per hectare; about 1×10¹² to about 1×10¹³ colony forming units (CFU) per hectare, about 1×10¹³ to about 1×10¹⁴ colony forming units (CFU) per hectare, about 1×10¹⁴ to about 1×10¹⁵ colony forming units (CFU) per hectare, about 1×10¹⁵ to about 1×10¹⁶ colony forming units (CFU) per hectare, about 1×10¹⁶ to about 1×10¹⁷ colony forming units (CFU) per hectare; about 1×10⁴ to about 1×10¹⁴ colony forming units (CFU) per hectare; about 1×10⁵ to about 1×10¹³ colony forming units (CFU) per hectare; about 1×10⁶ to about 1×10¹² colony forming units (CFU) per hectare; about 1×10⁹ to about 1×10¹¹ colony forming units (CFU) per hectare; or about 1×10⁹ to about 1×10¹¹ colony forming units (CFU) per hectare. In other embodiments, for a field inoculation, the rate of bacterial strain provided herein or active variant thereof application is at least about 1×10⁴, about 1×10⁵, about 1×10⁶, about 1×10⁷, about 1×10⁸, about 1×10⁹, about 1×10¹⁰, about 1×10¹¹, about 1×10¹²1×10¹³, about 1×10¹⁴, 1×10¹⁵, about 1×10¹⁶, or about 1×10¹⁷ colony forming units (CFU) per hectare. In other embodiments, for a field inoculation, the rate of bacterial strain provided herein or active variant thereof application is at least 1×10⁷ to at least about 1×10¹² CFU/hectare. In specific embodiments, the bacterial strain provided herein or active variant thereof applied comprises the strain deposited as AIP27511, AIP35174, AIP25773, AIP15251, AIP61892, AIP79428, AIP14931, AIP39589, or AIP36895, or an active derivative of any thereof, or a spore, or a forespore or a combination of cells, forespores and/or spores from any one of AIP27511, AIP35174, AIP25773, AIP15251, AIP61892, AIP79428, AIP14931, AIP39589, or AIP36895, or an active derivative of any thereof.

Any appropriate agricultural application rate for a biocide can be applied in combination with the bacterial strain provided herein or active variant thereof disclosed herein. Methods to assay for the effective amount of the bacterial strain provided herein or active variant thereof include, for example, any statistically significant increase in the control of the pathogen or pest targeted by the biocide. Methods to assay for such control are known. Moreover, a statistically significant increase in the control of plant health, yield and/or growth that occurs upon application of an effective amount of the bacterial strain provided herein or active variant thereof when compared to the plant health, yield and/or growth that occurs when no bacterial strain provided herein or active variant thereof is applied.

Further provided is a method for controlling or inhibiting the growth of a plant pathogen that causes plant disease by applying a composition comprising at least one bacterial strain provided herein or active variant thereof provided herein (i.e., AIP27511, AIP35174, AIP25773, AIP15251, AIP61892, AIP79428, AIP14931, AIP39589, or AIP36895, or an active variant or any thereof, or a spore, or a forespore or a combination of cells, forespores and/or spores from any one of AIP27511, AIP35174, AIP25773, AIP15251, AIP61892, AIP79428, AIP14931, AIP39589, or AIP36895, or an active variant any of thereof). By “applying” is intended contacting an effective amount of the bacterial strain provided herein or active variant thereof to a plant, area of cultivation, seed and/or weed with one or more of the bacterial strain provided herein or active variant thereof so that a desired effect is achieved. Furthermore, the application of the bacterial strain provided herein or active variant thereof can occur prior to the planting of the crop (for example, to the soil, the seed, or the plant). In a specific embodiment, the application of the bacterial strain provided herein or active variant thereof is a foliar application. Therefore, a further embodiment of the invention provides a method for controlling or inhibiting the growth of a plant pathogen by applying the population of bacterial strain provided herein or active variant thereof to an environment in which the plant pathogen may grow. The application may be to the plant, to parts of the plant, to the seeds of the plants to be protected, or to the soil in which the plant to be protected are growing or will grow. Application to the plant or plant parts may be before or after harvest. Application to the seeds will be prior to planting of the seeds.

In some embodiments, an effective amount of at least one bacterial strain provided herein or active variant thereof provided herein is used as a foliar application to control or inhibit growth of one or more pathogens selected from the group consisting of Alternaria spp., Alternaria solani, Colletotrichum spp., Mycosphaerella spp., Phomopsis spp., Cercospora spp., Botrytis spp., and Botrytis cinerea.

In other embodiments, an effective amount of at least one bacterial strain provided herein or active variant thereof provided herein is applied to the soil in which the plant to be protected are growing or will grow to control or inhibit growth of one or more pathogens selected from the group consisting of Rhizoctonia spp., Rhizoctonia solani, Fusarium spp., Sclerotium spp., Sclerotinia spp., Sclerotinia sclerotiorum, Phytopthora spp., and Pythium spp.

In some embodiments, an effective amount of at least one bacterial strain provided herein or active variant thereof provided herein is applied to the plant after harvest to control or inhibit growth of one or more pathogens selected from the group consisting of Monolinia spp., Penicillium spp., Botrytis ssp., and Botrytis cinerea.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species.

In specific embodiments, the application of the bacterial strain provided herein or active variant thereof (i.e., AIP27511, AIP35174, AIP25773, AIP15251, AIP61892, AIP79428, AIP14931, AIP39589, or AIP36895, or an active variant of any thereof, or a spore, or a forespore or a combination of cells, forespores and/or spores from any one of AIP27511, AIP35174, AIP25773, AIP15251, AIP61892, AIP79428, AIP14931, AIP39589, or AIP36895, or an active variant thereof) is applied to the leaves of a soybean plant. The timing of application can vary depending on the conditions and geographical location. In specific embodiments, the bacterial strain provided herein or active variant thereof is applied at the R1 (beginning flowering stage) of soybean development or may be applied earlier depending on ASR onset and the disease severity.

In other embodiments, the biocide to a crop, area of cultivation, or field it is intended that one or more of a particular field, plant crop, seed and/or weed is treated with one or more of the bacterial strain provided herein or active variant thereof and one or more biocide so that a desired effect is achieved.

Various methods are provided for controlling a plant pathogen that causes a plant disease in an area of cultivation containing a plant susceptible to the plant disease. The method comprises planting the area of cultivation with seeds or plants susceptible to the plant disease; and applying to the plant susceptible to the disease, the seed or the area of cultivation of the plant susceptible to the plant disease an effective amount of at least one bacterial strain provided herein or active variant thereof (i.e., AIP27511, AIP35174, AIP25773, AIP15251, AIP61892, AIP79428, AIP14931, AIP39589, or AIP36895, or an active derivative or any thereof, or a spore, or a forespore or a combination of cells, forespores and/or spores from any one of AIP27511, AIP35174, AIP25773, AIP15251, AIP61892, AIP79428, AIP14931, AIP39589, or AIP36895, or an active variant thereof), wherein the effective amount of the bacterial strain provided herein or active variant thereof controls the plant disease without significantly affecting the crop. In specific embodiments, the effective amount comprises at least about 10¹² to 10¹⁶ colony forming units (CFU) per hectare.

Further provided is a method for growing a plant susceptible to a plant disease. The method comprises applying to a plant susceptible to the disease, a seed, or an area of cultivation of the plant susceptible to the disease an effective amount of a composition comprising at least one bacterial strain provided herein or active variant thereof. In certain embodiments, the bacterial strain provided herein or active variant thereof may comprise at least one of AIP27511, AIP35174, AIP25773, AIP15251, AIP61892, AIP79428, AIP14931, AIP39589, or AIP36895, or an active variant of any thereof; or a spore, or a forespore or a combination of cells, forespores and/or spores from any one of AIP27511, AIP35174, AIP25773, AIP15251, AIP61892, AIP79428, AIP14931, AIP39589, or AIP36895, or an active variant of any thereof. Various effective amounts of bacterial strain provided herein or active variant thereof are disclosed elsewhere herein and in one, non-limiting example, the effective amount of the bacterial strain provided herein or active variant thereof comprises at least about 10¹² to 10¹⁶ colony forming units (CFU) per hectare.

Methods for increasing plant yield are provided. The “yield” of the plant refers to the quality and/or quantity of biomass produced by the plant. By “biomass” is intended any measured plant product. An increase in biomass production is any improvement in the yield of the measured plant product. An increase in yield can comprise any statistically significant increase including, but not limited to, at least a 1% increase, at least a 3% increase, at least a 5% increase, at least a 10% increase, at least a 20% increase, at least a 30%, at least a 50%, at least a 70%, at least a 100% or a greater increase in yield compared to a plant not exposed to the bacterial strain provided herein or active variant thereof. A method for increasing yield in a plant is also provided and comprises applying to a crop or an area of cultivation an effective amount of a composition comprising at least one bacterial strain comprising AIP27511, AIP35174, AIP25773, AIP15251, AIP61892, AIP79428, AIP14931, AIP39589, and AIP36895, or an active variant of any thereof, a spore or a forespore or a combination of cells, forespores and/or spores from any one of AIP27511, AIP35174, AIP25773, AIP15251, AIP61892, AIP79428, AIP14931, AIP39589, or AIP36895, or an active variant of any thereof, wherein said effective amount comprises at least about 10¹² to 10¹⁶ colony forming units (CFU) per hectare, and wherein said composition controls a plant pathogen, thereby increasing yield.

As used herein, an “area of cultivation” comprises any region in which one desires to grow a plant. Such areas of cultivations include, but are not limited to, a field in which a plant is cultivated (such as a crop field, a sod field, a tree field, a managed forest, a field for culturing fruits and vegetables, etc.), a greenhouse, a growth chamber, etc.

Further provided is a coated seed which comprises a seed and a coating on the seed, wherein the coating comprises at least one bacterial strain provided herein or active variant thereof. In certain embodiments, the bacterial strain provided herein or active variant thereof may comprise at least one of AIP27511, AIP35174, AIP25773, AIP15251, AIP61892, AIP79428, AIP14931, AIP39589, or AIP36895, or an active variant of any thereof; or a spore, or a forespore or a combination of cells, forespores and/or spores from any one of AIP27511, AIP35174, AIP25773, AIP15251, AIP61892, AIP79428, AIP14931, AIP39589, or AIP36895, or an active variant of any thereof. In certain embodiments, said bacterial strain provided herein or active variant thereof is present on the seed at about 10⁵ CFU/seed to about 10⁷ CFU/seed, at about 10⁴ CFU/seed to about 10⁸ CFU/seed, at about 10⁴ CFU/seed to about 10⁵ CFU/seed, at about 10⁵ CFU/seed to about 10⁶ CFU/seed, at about 10⁶ CFU/seed to about 10⁷ CFU/seed, or at about 10⁷ CFU/seed to about 10⁸ CFU/seed. The seed coating can be applied to any seed of interest (i.e., for a monocot or a dicot). Various plants of interest are disclosed elsewhere herein.

A seed coating can further comprise at least at least one nutrient, at least one herbicide or at least one pesticide, or at least one biocide. See, for example, US App Pub. 20040336049, 20140173979, and 20150033811.

In other embodiments, a plant of interest (i.e., plant susceptible to the plant disease) and/or the area of cultivation comprising the plant, can be treated with a combination of an effective amount of the bacterial strain provided herein or active variant thereof and an effective amount of a biocide. By “treated with a combination of” or “applying a combination of” a bacterial strain provided herein or active variant thereof and a biocide to a plant, area of cultivation or field it is intended that one or more of a particular field, plant, and/or weed is treated with an effective amount of one or more of the bacterial strain provided herein or active variant thereof and one or more biocide so that a desired effect is achieved. Furthermore, the application of one or both of the bacterial strain provided herein or active variant thereof and the biocide can occur prior to the planting of the crop (for example, to the soil, or the plant). Moreover, the application of the bacterial strain provided herein or active variant thereof and the biocide may be simultaneous or the applications may be at different times (sequential), so long as the desired effect is achieved.

In one non-limiting embodiment, the active variant comprises a bacterial strain provided herein that is resistance to one or more biocide. In specific embodiments, the bacterial strain provided herein or active variant thereof (i.e., AIP27511, AIP35174, AIP25773, AIP15251, AIP61892, AIP79428, AIP14931, AIP39589, or AIP36895, or an active variant of any thereof, or a spore, or a forespore or a combination of cells, forespores and/or spores from any one of AIP27511, AIP35174, AIP25773, AIP15251, AIP61892, AIP79428, AIP14931, AIP39589, or AIP36895, or an active variant of any thereof) is resistant to glyphosate. In such methods, a plant, crop, or area of cultivation is treated with a combination of an effective amount of the bacterial strain provided herein or active variant thereof that is resistant to glyphosate and an effective amount of glyphosate, wherein the effective amount of glyphosate is such as to selectively control weeds while the crop is not significantly damaged.

In another non-limiting embodiment, the active variant comprises a bacterial strain provided herein that is resistant to glufosinate. In such methods, a plant, crop, or area of cultivation is treated with a combination of an effective amount of the bacterial strain provided herein or active variant thereof that is resistant to glufosinate and an effective amount of glufosinate, wherein the effective amount of glufosinate is such as to selectively control weeds while the crop is not significantly damaged. In such embodiments, the effective amount of the bacterial strain provided herein or active variant thereof is sufficient to result in a statistically significant increase in plant health, yield, and/or growth when compared to the plant health, yield, and/or growth that occurs when the same concentration of a bacterial strain provided herein or active variant thereof that was not modified to be resistant to glufosinate is applied in combination with the effective amount of the glufosinate or active derivative thereof. In a further embodiment, the bacterial strain provided herein or active variant thereof comprises an effective amount of AIP27511, AIP35174, AIP25773, AIP15251, AIP61892, AIP79428, AIP14931, AIP39589, or AIP36895, or an active variant of any thereof, or a spore, or a forespore or a combination of cells, forespores and/or spores from any one of AIP27511, AIP35174, AIP25773, AIP15251, AIP61892, AIP79428, AIP14931, AIP39589, or AIP36895, or an active variant of any thereof.

V. Biocides for Use in Combination with the Bacterial Strains Provided Herein or Active Variant Thereof

As discussed elsewhere herein, the bacterial strain provided herein or active variant thereof can be used in combination with a biocide (i.e., a herbicide, fungicide, pesticide, or other crop protection chemical). In such instances, the bacterial strain provided herein or active variant thereof is compatible with the biocide of interest.

By “herbicide, fungicide, pesticide, or other crop protection chemical tolerance or herbicide, fungicide, pesticide, or other crop protection chemical resistance” is intended the ability of an organism (i.e., the plant and/or the bacterial strain provided herein or active variant thereof etc.) to survive and reproduce following exposure to a dose of the herbicide, fungicide, pesticide, or other crop protection chemical that is normally lethal to the wild type organism.

Herbicides that can be used in the various methods and compositions discloses herein include glyphosate, ACCase inhibitors (Arloxyphenoxy propionate (FOPS)); ALS inhibitors (Sulfonylurea (SU)), Imidazonlinone (IMI), Pyrimidines (PM)); microtubule protein inhibitor (Dinitroaniline (DNA)); synthetic auxins (Phenoxy (P)), Benzoic Acid (BA), Carboxylic acid (CA)); Photosystem II inhibitor (Triazine (TZ)), Triazinone (TN), Nitriles (NT), Benzothiadiazinones (BZ), Ureas (US)); EPSP Synthase inhibitor (glycines (GC)); Glutamine Synthesis inhibitor (Phosphinic Acid (PA)); DOXP synthase inhibitor (Isoxazolidinone (IA)); HPPD inhibitor (Pyrazole (PA)), Triketone (TE)); PPO inhibitors (Diphenylether (DE), N-phenylphthalimide (NP) (Ary triazinone (AT)); VLFA inhibitors (chloroacetamide (CA)), Oxyacetamide (OA)); Photosystem I inhibitor (Bipyridyliums (BP)); and the like.

Pesticides that can be used in the various methods and compositions disclosed herein include imidacloprid clothianidin, arylpyrazole compounds (WO2007103076); organophosphates, phenyl pyrazole, pyrethoids caramoyloximes, pyrazoles, amidines, halogenated hydrocarbons, carbamates and derivatives thereof, terbufos, chloropyrifos, fipronil, chlorethoxyfos, telfuthrin, carbofuran, imidacloprid, tebupirimfos (U.S. Pat. No. 5,849,320).

Fungicides that can be used in the various methods and compositions disclosed herein include aliphatic nitrogen fungicides (butylamine, cymoxanil, dodicin, dodine, guazatine, iminoctadine); amide fungicides (benzovindiflupyr, carpropamid, chloraniformethan, cyflufenamid, diclocymet, diclocymet, dimoxystrobin, fenaminstrobin, fenoxanil, flumetover, furametpyr, isofetamid, isopyrazam, mandestrobin, mandipropamid, metominostrobin, orysastrobin, penthiopyrad, prochloraz, quinazamid, silthiofam, triforine); acylamino acid fungicides (benalaxyl, benalaxyl-M, furalaxyl, metalaxyl, metalaxyl-M, pefurazoate, valifenalate); anilide fungicides (benalaxyl, benalaxyl-M, bixafen, boscalid, carboxin, fenhexamid, fluxapyroxad, isotianil, metalaxyl, metalaxyl-M, metsulfovax, ofurace, oxadixyl, oxycarboxin, penflufen, pyracarbolid, sedaxane, thifluzamide, tiadinil, vanguard); benzanilide fungicides (benodanil, flutolanil, mebenil, mepronil, salicylanilide, tecloftalam); furanilide fungicides (fenfuram, furalaxyl, furcarbanil, methfuroxam); sulfonanilide fungicides (flusulfamide); benzamide fungicides (benzohydroxamic acid, fluopicolide, fluopyram, tioxymid, trichlamide, zarilamid, zoxamide); furami de fungicides (cyclafuramid, furmecyclox); phenylsulfamide fungicides (dichlofluanid, tolylfluanid); sulfonamide fungicides (amisulbrom, cyazofamid); valinamide fungicides (benthiavalicarb, iprovalicarb); antibiotic fungicides (aureofungin, blasticidin-S, cycloheximide, griseofulvin, kasugamycin, moroxydine, natamycin, polyoxins, polyoxorim, streptomycin, validamycin); strobilurin fungicides (fluoxastrobin, mandestrobin); methoxyacrylate strobilurin fungicides (azoxystrobin, bifujunzhi, coumoxystrobin, enoxastrobin, flufenoxystrobin, jiaxiangjunzhi, picoxystrobin, pyraoxystrobin); methoxycarbanilate strobilurin fungicides (pyraclostrobin, pyrametostrobin, triclopyricarb); methoxyiminoacetamide strobilurin fungicides (dimoxystrobin, fenaminstrobin, metominostrobin, orysastrobin); methoxyiminoacetate strobilurin fungicides (kresoxim-methyl, trifloxystrobin); aromatic fungicides (biphenyl, chlorodinitronaphthalenes, chloroneb, chlorothalonil, cresol, dicloran, fenjuntong, hexachlorobenzene, pentachlorophenol, quintozene, sodium pentachlorophenoxide, tecnazene, trichlorotrinitrobenzenes); arsenical fungicides (asomate, urbacide); aryl phenyl ketone fungicides (metrafenone, pyriofenone); benzimidazole fungicides (albendazole, benomyl, carbendazim, chlorfenazole, cypendazole, debacarb, fuberidazole, mecarbinzid, rabenzazole, thiabendazole); benzimidazole precursor fungicides (furophanate, thiophanate, thiophanate-methyl); benzothiazole fungicides (bentaluron, benthiavalicarb, benthiazole, chlobenthiazone, probenazole); botanical fungicides (allicin, berberine, carvacrol, carvone, osthol, sanguinarine, santonin); bridged diphenyl fungicides (bithionol, dichlorophen, diphenylamine, hexachlorophene, parinol); carbamate fungicides (benthiavalicarb, furophanate, iodocarb, iprovalicarb, picarbutrazox, propamocarb, pyribencarb, thiophanate, thiophanate-methyl, tolprocarb); benzimidazolylcarbamate fungicides (albendazole, benomyl, carbendazim, cypendazole, debacarb, mecarbinzid); carbanilate fungicides (diethofencarb, pyraclostrobin, pyrametostrobin, triclopyricarb); conazole fungicides, conazole fungicides (imidazoles) (climbazole, clotrimazole, imazalil, oxpoconazole, prochloraz, triflumizole); conazole fungicides (triazoles) (azaconazole, bromuconazole, cyproconazole, diclobutrazol, difenoconazole, diniconazole, diniconazole-M, epoxiconazole, etaconazole, fenbuconazole, fluquinconazole, flusilazole, flutriafol, furconazole, furconazole-cis, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole, uniconazole, uniconazole-P); copper fungicides (acypetacs-copper, Bordeaux mixture, Burgundy mixture, Cheshunt mixture, copper acetate, copper carbonate, basic, copper hydroxide, copper naphthenate, copper oleate, copper oxychloride, copper silicate, copper sulfate, copper sulfate, basic, copper zinc chromate, cufraneb, cuprobam, cuprous oxide, mancopper, oxine-copper, saisentong, thiodiazole-copper); cyanoacrylate fungicides (benzamacril, phenamacril); dicarboximide fungicides (famoxadone, fluoroimide); dichlorophenyl dicarboximide fungicides (chlozolinate, dichlozoline, iprodione, isovaledione, myclozolin, procymidone, vinclozolin); phthalimide fungicides (captafol, captan, ditalimfos, folpet, thiochlorfenphim); dinitrophenol fungicides (binapacryl, dinobuton, dinocap, dinocap-4, dinocap-6, meptyldinocap, dinocton, dinopenton, dinosulfon, dinoterbon, DNOC); dithiocarbamate fungicides (amobam, asomate, azithiram, carbamorph, cufraneb, cuprobam, disulfiram, ferbam, metam, nabam, tecoram, thiram, urbacide, ziram); cyclic dithiocarbamate fungicides (dazomet, etem, milneb); polymeric dithiocarbamate fungicides (mancopper, mancozeb, maneb, metiram, polycarbamate, propineb, zineb); dithiolane fungicides (isoprothiolane, saijunmao); fumigant fungicides (carbon disulfide, cyanogen, dithioether, methyl bromide, methyl iodide, sodium tetrathiocarbonate); hydrazide fungicides (benquinox, saijunmao); imidazole fungicides (cyazofamid, fenamidone, fenapanil, glyodin, iprodione, isovaledione, pefurazoate, triazoxide); conazole fungicides (imidazoles) (climbazole, clotrimazole, imazalil, oxpoconazole, prochloraz, triflumizole); inorganic fungicides (potassium azide, potassium thiocyanate, sodium azide, sulfur, see also copper fungicides, see also inorganic mercury fungicides); mercury fungicides; inorganic mercury fungicides (mercuric chloride, mercuric oxide, mercurous chloride); organomercury fungicides ((3-ethoxypropyl)mercury bromide, ethylmercury acetate, ethylmercury bromide, ethylmercury chloride, ethylmercury 2,3-dihydroxypropyl mercaptide, ethylmercury phosphate, N-(ethylmercury)-p-toluenesulphonanilide, hydrargaphen, 2-methoxyethylmercury chloride, methylmercury benzoate, methylmercury dicyandiamide, methylmercury pentachlorophenoxide, 8-phenylmercurioxyquinoline, phenylmercuriurea, phenylmercury acetate, phenylmercury chloride, phenylmercury derivative of pyrocatechol, phenylmercury nitrate, phenylmercury salicylate, thiomersal, tolylmercury acetate); morpholine fungicides (aldimorph, benzamorf, carbamorph, dimethomorph, dodemorph, fenpropimorph, flumorph, tridemorph); organophosphorus fungicides (ampropylfos, ditalimfos, EBP, edifenphos, fosetyl, hexylthiofos, inezin, iprobenfos, izopamfos, kejunlin, phosdiphen, pyrazophos, tolclofos-methyl, triamiphos); organotin fungicides (decafentin, fentin, tributyltin oxide); oxathiin fungicides (carboxin, oxycarboxin); oxazole fungicides (chlozolinate, dichlozoline, drazoxolon, famoxadone, hymexazol, metazoxolon, myclozolin, oxadixyl, oxathiapiprolin, pyrisoxazole, vinclozolin); polysulfide fungicides (barium polysulfide, calcium poly sulfide, potassium polysulfide, sodium polysulfide); pyrazole fungicides (benzovindiflupyr, bixafen, fenpyrazamine, fluxapyroxad, furametpyr, isopyrazam, oxathiapiprolin, penflufen, penthiopyrad, pyraclostrobin, pyrametostrobin, pyraoxystrobin, rabenzazole, sedaxane); pyridine fungicides (boscalid, buthiobate, dipyrithione, fluazinam, fluopicolide, fluopyram, parinol, picarbutrazox, pyribencarb, pyridinitril, pyrifenox, pyrisoxazole, pyroxychlor, pyroxyfur, triclopyricarb); pyrimidine fungicides (bupirimate, diflumetorim, dimethirimol, ethirimol, fenarimol, ferimzone, nuarimol, triarimol); anilinopyrimidine fungicides (cyprodinil, mepanipyrim, pyrimethanil); pyrrole fungicides (dimetachlone, fenpiclonil, fludioxonil, fluoroimide); quaternary ammonium fungicides (berberine, sanguinarine); quinoline fungicides (ethoxyquin, halacrinate, 8-hydroxyquinoline sulfate, quinacetol, quinoxyfen, tebufloquin); quinone fungicides (chloranil, dichlone, dithianon); quinoxaline fungicides (chinomethionat, chlorquinox, thioquinox); thiadiazole fungicides (etridiazole, saisentong, thiodiazole-copper, zinc thiazole); thiazole fungicides (ethaboxam, isotianil, metsulfovax, octhilinone, oxathiapiprolin, thiabendazole, thifluzamide); thiazolidine fungicides (flutianil, thiadifluor); thiocarbamate fungicides (methasulfocarb, prothiocarb); thiophene fungicides (ethaboxam, isofetamid, silthiofam); triazine fungicides (anilazine); triazole fungicides (amisulbrom, bitertanol, fluotrimazole, triazbutil); conazole fungicides (triazoles) (azaconazole, bromuconazole, cyproconazole, diclobutrazol, difenoconazole, diniconazole, diniconazole-M, epoxiconazole, etaconazole, fenbuconazole, fluquinconazole, flusilazole, flutriafol, furconazole, furconazole-cis, hexaconazole, huanjunzuo, imibenconazole, ipconazole, metconazole, myclobutanil, penconazole, propiconazole, prothioconazole, quinconazole, simeconazole, tebuconazole, tetraconazole, triadimefon, triadimenol, triticonazole, uniconazole, uniconazole-P); triazolopyrimidine fungicides (ametoctradin); urea fungicides (bentaluron, pencycuron, quinazamid); zinc fungicides (acypetacs-zinc, copper zinc chromate, cufraneb, mancozeb, metiram, polycarbamate, polyoxorim-zinc, propineb, zinc naphthenate, zinc thiazole, zinc trichlorophenoxide, zineb, ziram); unclassified fungicides (acibenzolar, acypetacs, allyl alcohol, benzalkonium chloride, bethoxazin, bromothalonil, chitosan, chloropicrin, DBCP, dehydroacetic acid, diclomezine, diethyl pyrocarbonate, ethylicin, fenaminosulf, fenitropan, fenpropidin, formaldehyde, furfural, hexachlorobutadiene, methyl isothiocyanate, nitrostyrene, nitrothal-isopropyl, OCH, pentachlorophenyl laurate, 2-phenylphenol, phthalide, piperalin, propamidine, proquinazid, pyroquilon, sodium orthophenylphenoxide, spiroxamine, sultropen, thicyofen, tricyclazole), or mefenoxam.

Non-limiting embodiments of the invention include:

1. A composition comprising:

(a) at least one of bacterial strain AIP27511, AIP35174, AIP25773, AIP15251, AIP61892, AIP79428, AIP14931, AIP39589, or AIP36895, or an active variant of any thereof, wherein the active variant comprises a bacterial strain having a genome within a Mash distance of about 0.015; and/or

(b) at least one of a spore, or a forespore, or a combination of cells, forespores and/or spores from any one of AIP27511, AIP35174, AIP25773, AIP15251, AIP61892, AIP79428, AIP14931, AIP39589, or AIP36895, or an active variant of any thereof, wherein the active variant comprises a bacterial strain having a genome within a Mash distance of about 0.015;

wherein said bacterial strain, spore, or a forespore, or a combination of cells, forespores and/or spores or the active variant of any thereof is present at about 10⁵ CFU/gram to about 10¹² CFU/gram or at about 10⁵ CFU/ml to about 10¹² CFU/ml, and wherein an effective amount of said bacterial strain composition improves an agronomic trait of interest of the plant or controls a plant pathogen that causes a plant disease.

2. The composition of embodiment 1, wherein the plant disease is a fungal plant disease.

3. The composition of embodiment 1 or 2, wherein the plant disease is Asian Soybean Rust (ASR).

4. The composition of any of embodiments 1-3, wherein said bacterial strain or the active variant thereof is present at about 10⁵ CFU/gram to about 10¹⁰ CFU/gram or at about 10⁵ CFU/ml to about 10¹⁰ CFU/ml.

5. The composition of any of embodiments 1-4, wherein said composition comprises a cell paste.

6. The composition of any one of embodiments 1-5, wherein said composition comprises a wettable powder.

7. The composition of any one of embodiments 1-6, wherein said plant pathogen comprises at least one fungal pathogen.

8. The composition of embodiment 7, wherein said plant pathogen comprises one or more fungal pathogens selected from the group consisting of Botrytis cinerea, Cersospora spp, Cercospora sojina, Cercospora beticola, Alternaria solani, Rhizoctonia solani, Blumeria graminis f. sp. Tritici, Erysiphe necator, Podosphaera xanthii, Golovinomyces cichoracearum, Erysiphe lagerstroemiae, Sphaerotheca pannosa, Colletotrichum cereale, Apiognornonia errabunda, Apiognomonia veneta, Colletotrichum gloeosporiodes, Discula fraxinea, Plasmopara viticola, Pseudoperonospora cubensis, Peronospora belbahrii, Bremia lactucae, Peronospora lamii, Plasmopara obduscens, Pythium cryptoirregulare, Pythium aphanidermatum, Pythium irregulare, Pythium sylvaticum, Pythium myriotylum, Pythium ultimum, Phytophthora capsici, Phytophthora nicotianae, Phytophthora infestans, Phytophthora tropicalis, Phytophthora sojae, Fusarium graminearum, Fusarium solani, Fusarium oxysporum, Fusarium graminicola, Gibberella zeae, Colletotrichum graminicola, Phakopsora sp., Phakopsora meibomiae, Phakopsora pachyrizi, Puccinia triticina, Puccinia recondita, Puccinia striiformis, Puccinia graminis, Puccinia spp., Venturia inaequalis, Verticillium spp, Erwinia amylovora, Monilinia fructicola, Monilinia lax, and Monilinia fructigena.

9. The composition of embodiment 8, wherein said plant pathogen comprises one or more fungal pathogens selected from the group consisting of Botrytis cinerea, Cercospora sojina, Alternaria solani, Rhizoctonia solani, Erysiphe necator, Podosphaera xanthii, Colletotrichum cereal, Plasmopara viticola, Peronospora belbahrii, Pythium aphanidermatum, Pythium sylvaticum, Pythium myriotylum, Pythium ultimurn, Phytophthora nicotianae, Phytophthora infestans, Phytophthora tropicalis, Phytophthora sojae, Fusarium graminearum, Fusarium solani, Phakopsora pachyrizi, and Venturia inaequalisa.

10. The composition of embodiment 8, wherein said plant pathogen comprises Phakopsora pachyrhizi or Phakopsora meibomiae.

11. The composition of embodiment 10, wherein said pathogen comprises Phakopsora pachyrhizi.

12. A composition comprising a cell paste comprising:

(a) at least one of bacterial strain AIP27511, AIP35174, AIP25773, AIP15251, AIP61892, AIP79428, AIP14931, AIP39589, or AIP36895, or an active variant of any thereof, wherein the active variant comprises a bacterial strain having a genome within a Mash distance of about 0.015; and/or,

(b) at least one of a spore, or a forespore, or a combination of cells, forespores and/or spores from any one of AIP27511, AIP35174, AIP25773, AIP15251, AIP61892, AIP79428, AIP14931, AIP39589, or AIP36895, or an active variant of any thereof, wherein the active variant comprises a bacterial strain having a genome within a Mash distance of about 0.015;

wherein an effective amount of said bacterial strain composition improves an agronomic trait of interest of the plant or controls a plant pathogen that causes a plant disease.

13. The composition of embodiment 12, wherein the plant disease is a fungal plant disease.

14. The composition of any one of embodiments 12-13, wherein the plant disease is Asian Soybean Rust.

15. The composition of any one of embodiments 12-14, wherein the plant pathogen comprises at least one fungal pathogen.

16. The composition of embodiment 15, wherein said plant pathogen comprises one or more fungal pathogens selected from the group consisting of Botrytis cinerea, Cersospora spp, Cercospora sojina, Cercospora beticola, Alternaria solani, Rhizoctonia solani, Blumeria graminis f. sp. Tritici, Erysiphe necator, Podosphaera xanthii, Golovinomyces cichoracearum, Erysiphe lagerstroemiae, Sphaerotheca pannosa, Colletotrichum cereale, Apiognomonia errabunda, Apiognomonia veneta, Colletotrichum gloeosporiodes, Discula fraxinea, Plasmopara viticola, Pseudoperonospora cubensis, Peronospora belbahrii, Bremia lactucae, Peronospora lamii, Plasmopara obduscens, Pythium cryptoirregulare, Pythium aphanidermatum, Pythium irregulare, Pythium sylvaticum, Pythium myriotylum, Pythium ultimum, Phytophthora capsici, Phytophthora nicotianae, Phytophthora infestans, Phytophthora tropicalis, Phytophthora sojae, Fusarium graminearum, Fusarium solani, Fusarium oxysporum, Fusarium graminicola, Gibberella zeae, Colletotrichum graminicola, Phakopsora sp., Phakopsora meibomiae, Phakopsora pachyrizi, Puccinia triticina, Puccinia recondita, Puccinia striifbrmis, Puccinia graminis, Puccinia spp., Venturia inaequalis, Verticillium spp, Erwinia amylovora, Monilinia fructicola, Monilinia lax, and Monilinia fructigena.

17. The composition of embodiment 16, wherein said plant pathogen comprises one or more fungal pathogens selected from the group consisting of Botrytis cinerea, Cercospora sojina, Alternaria solani, Rhizoctonia solani, Erysiphe necator, Podosphaera xanthii, Colletotrichum cereal, Plasmopara viticola, Peronospora belbahrii, Pythium aphanidermatum, Pythium sylvaticum, Pythium myriotylum, Pythium ultimum, Phytophthora nicotianae, Phytophthora infestans, Phytophthora tropicalis, Phytophthora sojae, Fusarium graminearum, Fusarium solani, Phakopsora pachyrizi, and Venturia inaequalisa.

18. The composition of embodiment 16, wherein said plant pathogen comprises Phakopsora pachyrhizi or Phakopsora meibomiae.

19. The composition of embodiment 18, wherein said plant pathogen comprises Phakopsora pachyrhizi.

20. A composition comprising a wettable power comprising

(a) at least one of bacterial strain AIP27511, AIP35174, AIP25773, AIP15251, AIP61892, AIP79428, AIP14931, AIP39589, or AIP36895, or an active variant of any thereof, wherein the active variant comprises a bacterial strain having a genome within a Mash distance of about 0.015; and/or,

(b) at least one of a spore, or a forespore, or a combination of cells, forespores and/or spores from any one of AIP27511, AIP35174, AIP25773, AIP15251, AIP61892, AIP79428, AIP14931, AIP39589, or AIP36895, or an active variant of any thereof, wherein the active variant comprises a bacterial strain having a genome within a Mash distance of about 0.015;

wherein an effective amount of said bacterial strain composition improves an agronomic trait of interest of the plant or controls a plant pathogen that causes a plant disease.

21. The composition of embodiment 20, wherein the plant disease is a fungal plant disease.

22. The composition of embodiment 20 or 21, wherein the plant pathogen comprises at least one fungal pathogen.

23. The composition of embodiment 22, wherein the said plant pathogen comprises one or more fungal pathogens selected from the group consisting of Botrytis cinerea, Cersospora spp, Cercospora sojina, Cercospora beticola, Alternaria solani, Rhizoctonia solani, Blumeria graminis f. sp. Tritici, Erysiphe necator, Podosphaera xanthii, Golovinomyces cichoracearum, Erysiphe lagerstroemiae, Sphaerotheca pannosa, Colletotrichum cereale, Apiognomonia errabunda, Apiognomonia veneta, Colletotrichum gloeosporiodes, Discula fraxinea, Plasmopara viticola, Pseudoperonospora cubensis, Peronospora belbahrii, Bremia lactucae, Peronospora lamii, Plasmopara obduscens, Pythium cryptoirregulare, Pythium aphanidermatum, Pythium irregulare, Pythium sylvaticum, Pythium myriotylum, Pythium ultimum, Phytophthora capsici, Phytophthora nicotianae, Phytophthora infestans, Phytophthora tropicalis, Phytophthora sojae, Fusarium graminearum, Fusarium solani, Fusarium oxysporum, Fusarium graminicola, Gibberella zeae, Colletotrichum graminicola, Phakopsora sp., Phakopsora meibomiae, Phakopsora pachyrizi, Puccinia triticina, Puccinia recondita, Puccinia striiformis, Puccinia graminis, Puccinia spp., Venturia inaequalis, Verticillium spp, Erwinia amylovora, Monilinia fructicola, Monilinia lax, and Monilinia fructigena.

24. The composition of embodiment 23, wherein said plant pathogen comprises one or more fungal pathogens selected from the group consisting of Botrytis cinerea, Cercospora sojina, Alternaria solani, Rhizoctonia solani, Erysiphe necator, Podosphaera xanthii, Colletotrichum cereal, Plasmopara viticola, Peronospora belbahrii, Pythium aphanidermatum, Pythium sylvaticum, Pythium myriotylum, Pythium ultimum, Phytophthora nicotianae, Phytophthora infestans, Phytophthora tropicalis, Phytophthora sojae, Fusarium graminearum, Fusarium solani, Phakopsora pachyrizi, and Venturia inaequalisa.

25. The composition of embodiment 23, wherein said plant pathogen comprises Phakopsora pachyrhizi or Phakopsora meibomiae.

26. The composition of embodiment 25, wherein said plant pathogen comprises Phakopsora pachyrhizi.

27. The composition of any one of embodiments 20-26, wherein said active variant is resistant to at least one herbicide, fungicide, pesticide, or other crop protection chemical.

28. The composition of embodiment 27, wherein said active variant is selected under herbicide, fungicide, pesticide, or other crop protection chemical pressure and is resistant to said herbicide, fungicide, pesticide, or other crop protection chemical.

29. The composition of any one of embodiments 27-29, wherein said active variant has been transformed with a herbicide resistance gene rendering the bacterial strain provided herein or active variant thereof herbicide resistant, and wherein said bacterial strain controls a plant pathogen that causes a plant disease.

30. The composition of embodiment 29, wherein the plant pathogen causes ASR.

31. The composition of any one of embodiments 27-30, wherein said herbicide is selected from the group consisting of glyphosate, glufosinate (glutamine synthase inhibitor), sulfonylurea and imidazolinone herbicides (branched chain amino acid synthesis inhibitors).

32. An isolated biologically pure culture of a bacterial strain comprising:

(a) AIP27511, AIP35174, AIP25773, AIP15251, AIP61892, AIP79428, AIP14931, AIP39589, or AIP36895, or an active variant of any thereof, wherein the active variant comprises a bacterial strain having a genome within a Mash distance of about 0.015; or,

(b) a spore, or a forespore, or a combination of cells, forespores and/or spores from any one of AIP27511, AIP35174, AIP25773, AIP15251, AIP61892, AIP79428, AIP14931, AIP39589, or AIP36895, or an active variant of any thereof, wherein the active variant comprises a bacterial strain having a genome within a Mash distance of about 0.015.

33. The isolated biologically pure culture of embodiment 33, wherein said bacterial strain is resistant to a biocide selected from a herbicide, a fungicide, a pesticide, or a crop protection chemical, wherein said culture is produced by growing in the presence of said biocide, and wherein said bacterial strain controls a pathogen that causes a plant disease.

34. The isolated biologically pure culture of embodiment 33, wherein said biologically pure culture is able to grow in the presence of glyphosate.

35. The isolated biologically pure culture of any one of embodiments 33-34, wherein the plant disease is a fungal plant disease.

36. The isolated biologically pure culture of embodiment 35, wherein the plant disease is ASR.

37. The isolated biologically pure culture of any one of embodiments 33-36, wherein the plant pathogen comprises at least one fungal pathogen.

38. The isolated biologically pure culture of embodiment 37, wherein said plant pathogen comprises one or more fungal pathogens selected from the group consisting of Botrytis cinerea, Cersospora spp, Cercospora sojina, Cercospora beticola, Alternaria solani, Rhizoctonia solani, Blumeria graminis f. sp. Tritici, Erysiphe necator, Podosphaera xanthii, Golovinomyces cichoracearum, Erysiphe lagerstroemiae, Sphaerotheca pannosa, Colletotrichum cereale, Apiognomonia errabunda, Apiognomonia veneta, Colletotrichum gloeosporiodes, Discula fraxinea, Plasmopara viticola, Pseudoperonospora cubensis, Peronospora belbahrii, Bremia lactucae, Peronospora lamii, Plasmopara obduscens, Pythium cryptoirregulare, Pythium aphanidermatum, Pythium irregulare, Pythium sylvaticum, Pythium myriotylum, Pythium ultimum, Phytophthora capsici, Phytophthora nicotianae, Phytophthora infestans, Phytophthora tropicalis, Phytophthora sojae, Fusarium graminearum, Fusarium solani, Fusarium oxysporum, Fusarium graminicola, Gibberella zeae, Colletotrichum graminicola, Phakopsora sp., Phakopsora meibomiae, Phakopsora pachyrizi, Puccinia triticina, Puccinia recondita, Puccinia striiformis, Puccinia graminis, Puccinia spp., Venturia inaequalis, Verticillium spp, Erwinia amylovora, Monilinia fructicola, Monilinia lax, and Monilinia fructigena.

39. The isolated biologically pure culture of embodiment 38, wherein said plant pathogen comprises one or more fungal pathogens selected from the group consisting of Botrytis cinerea, Cercospora sojina, Alternaria solani, Rhizoctonia solani, Erysiphe necator, Podosphaera xanthii, Colletotrichum cereal, Plasmopara viticola, Peronospora belbahrii, Pythium aphanidermatum, Pythium sylvaticum, Pythium myriotylum, Pythium ultimum, Phytophthora nicotianae, Phytophthora infestans, Phytophthora tropicalis, Phytophthora sojae, Fusarium graminearum, Fusarium solani, Phakopsora pachyrizi, and Venturia inaequalisa.

40. The isolated biologically pure culture of embodiment 38, wherein said plant pathogen comprises Phakopsora pachyrhizi or Phakopsora meibomiae.

41. The isolated biologically pure culture of embodiment 40, wherein said plant pathogen comprises Phakopsora pachyrhizi.

42. A bacterial culture grown from

(a) AIP27511, AIP35174, AIP25773, AIP15251, AIP61892, AIP79428, AIP14931, AIP39589, or AIP36895, or an active variant of any thereof, wherein the active variant comprises a bacterial strain having a genome within a Mash distance of about 0.015; or,

(b) a spore, or a forespore, or a combination of cells, forespores and/or spores from any one of AIP27511, AIP35174, AIP25773, AIP15251, AIP61892, AIP79428, AIP14931, AIP39589, or AIP36895, or an active variant of any thereof, wherein the active variant comprises a bacterial strain having a genome within a Mash distance of about 0.015;

wherein said bacterial culture has antipathogenic activity against a plant pathogen that causes a plant disease and is able to grow in the presence of glufosinate or an effective amount of said bacterial culture improves an agronomic trait of interest of the plant.

43. The bacterial culture of embodiment 42, wherein the plant disease is a fungal plant disease.

44. The bacterial culture of embodiment 43, wherein the plant disease is ASR.

45. The bacterial culture of any one of embodiments 42-44, wherein the plant pathogen comprises at least one fungal pathogen.

46. The bacterial culture of embodiment 45, wherein said plant pathogen comprises one or more fungal pathogens selected from the group consisting of Botrytis cinerea, Cersospora spp, Cercospora sojina, Cercospora beticola, Alternaria solani, Rhizoctonia solani, Blumeria graminis f. sp. Tritici, Erysiphe necator, Podosphaera xanthii, Golovinomyces cichoracearum, Erysiphe lagerstroemiae, Sphaerotheca pannosa, Colletotrichum cereale, Apiognomonia errabunda, Apiognomonia veneta, Colletotrichum gloeosporiodes, Discula fraxinea, Plasmopara viticola, Pseudoperonospora cubensis, Peronospora belbahrii, Bremia lactucae, Peronospora lamii, Plasmopara obduscens, Pythium cryptoirregulare, Pythium aphanidermatum, Pythium irregulare, Pythium sylvaticum, Pythium myriotylum, Pythium ultimum, Phytophthora capsici, Phytophthora nicotianae, Phytophthora infestans, Phytophthora tropicalis, Phytophthora sojae, Fusarium graminearum, Fusarium solani, Fusarium oxysporum, Fusarium graminicola, Gibberella zeae, Colletotrichum graminicola, Phakopsora sp., Phakopsora meibomiae, Phakopsora pachyrizi, Puccinia triticina, Puccinia recondita, Puccinia striiformis, Puccinia graminis, Puccinia spp., Venturia inaequalis, Verticillium spp, Erwinia amylovora, Monilinia fructicola, Monilinia lax, and Monilinia fructigena.

47. The bacterial culture of embodiment 46, wherein said plant pathogen comprises one or more fungal pathogens selected from the group consisting of Botrytis cinerea, Cercospora sojina, Alternaria solani, Rhizoctonia solani, Erysiphe necator, Podosphaera xanthii, Colletotrichum cereal, Plasmopara viticola, Peronospora belbahrii, Pythium aphanidermatum, Pythium sylvaticum, Pythium myriotylum, Pythium ultimum, Phytophthora nicotianae, Phytophthora infestans, Phytophthora tropicalis, Phytophthora sojae, Fusarium graminearum, Fusarium solani, Phakopsora pachyrizi, and Venturia inaequalisa.

48. The bacterial culture of embodiment 46, wherein said plant pathogen comprises Phakopsora pachyrhizi or Phakopsora meibomiae.

49. The bacterial culture of embodiment 48, wherein said plant pathogen comprises Phakopsora pachyrhizi.

50. A method for growing a plant susceptible to a plant disease or improving a agronomic trait of interest in a plant comprising applying to the plant

(a) an effective amount of at least one of bacterial strain AIP27511, AIP35174, AIP25773, AIP15251, AIP61892, AIP79428, AIP14931, AIP39589, or AIP36895, or an active variant of any thereof wherein the active variant comprises a bacterial strain having a genome within a Mash distance of about 0.015; and/or,

(b) an effective amount of at least one of a spore, or a forespore, or a combination of cells, forespores and/or spores from any one of AIP27511, AIP35174, AIP25773, AIP15251, AIP61892, AIP79428, AIP14931, AIP39589 or AIP36895 or an active variant of any thereof, wherein the active variant comprises a bacterial strain having a genome within a Mash distance of about 0.015; wherein said effective amount comprises at least about 10¹² to 10¹⁶ colony forming units (CFU) per hectare, and wherein said effective amount controls a plant pathogen that causes the plant disease or improves the agronomic trait of interest.

51. The method of embodiment 50, wherein said method increases yield of the plant susceptible to the plant disease.

52. The method of embodiment 50 or 51, wherein the plant disease is a plant disease caused by a fungal pathogen.

53. The method of embodiment 52, wherein the plant disease is Asian Soybean Rust (ASR).

54. The method of any one of embodiments 50-53, wherein the plant pathogen comprises at least one fungal pathogen.

55. The method of embodiment 54, wherein said plant pathogen comprises one or more fungal pathogens selected from the group consisting of Botrytis cinerea, Cersospora spp, Cercospora sojina, Cercospora beticola, Alternaria solani, Rhizoctonia solani, Blumeria graminis f. sp. Tritici, Erysiphe necator, Podosphaera xanthii, Golovinomyces cichoracearum, Erysiphe lagerstroemiae, Sphaerotheca pannosa, Colletotrichum cereale, Apiognornonia errabunda, Apiognomonia veneta, Colletotrichum gloeosporiodes, Discula fraxinea, Plasmopara viticola, Pseudoperonospora cubensis, Peronospora belbahrii, Bremia lactucae, Peronospora lamii, Plasmopara obduscens, Pythium cryptoirregulare, Pythium aphanidermatum, Pythium irregulare, Pythium sylvaticum, Pythium myriotylum, Pythium ultimum, Phytophthora capsici, Phytophthora nicotianae, Phytophthora infestans, Phytophthora tropicalis, Phytophthora sojae, Fusarium graminearum, Fusarium solani, Fusarium oxysporum, Fusarium graminicola, Gibberella zeae, Colletotrichum graminicola, Phakopsora sp., Phakopsora meibomiae, Phakopsora pachyrizi, Puccinia triticina, Puccinia recondita, Puccinia striifbrmis, Puccinia graminis, Puccinia spp., Venturia inaequalis, Verticillium spp, Erwinia amylovora, Monilinia fructicola, Monilinia lax, and Monilinia fructigena.

56. The method of embodiment 55, wherein said plant pathogen comprises one or more fungal pathogens selected from the group consisting of Botrytis cinerea, Cercospora sojina, Alternaria solani, Rhizoctonia solani, Erysiphe necator, Podosphaera xanthii, Colletotrichum cereal, Plasmopara viticola, Peronospora belbahrii, Pythium aphanidermatum, Pythium sylvaticum, Pythium myriotylurn, Pythium ultimum, Phytophthora nicotianae, Phytophthora infestans, Phytophthora tropicalis, Phytophthora sojae, Fusarium graminearum, Fusarium solani, Phakopsora pachyrizi, and Venturia inaequalisa.

57. The method of embodiment 55, wherein said plant pathogen comprises Phakopsora pachyrhizi or Phakopsora meibomiae.

58. The method of embodiment 57, wherein said plant pathogen comprises Phakopsora pachyrhizi.

59. A method of controlling a plant pathogen that causes a plant disease in an area of cultivation comprising:

(a) planting the area of cultivation with seeds or plants susceptible to the plant disease; and

(b) applying to the plant susceptible to the plant disease an effective amount of at least one bacterial strain comprising

(i) AIP27511, AIP35174, AIP25773, AIP15251, AIP61892, AIP79428, AIP14931, AIP39589, or AIP36895, or an active variant of any thereof; or,

(ii) a spore, or a forespore, or a combination of cells, forespores and/or spores from any one of AIP27511, AIP35174, AIP25773, AIP15251, AIP61892, AIP79428, AIP14931, AIP39589, or AIP36895, or an active variant of any thereof, wherein the active variant comprises a bacterial strain having a genome within a Mash distance of about 0.015; and wherein said effective amount comprises at least about 10¹² to 10¹⁶ colony forming units (CFU) per hectare.

60. The method of embodiment 59, wherein said plant is susceptible to a fungal plant disease. 61. The method of embodiment 60, wherein said plant is susceptible to Asian Soybean Rust (ASR).

62. The method of embodiment 61, where said plant susceptible to ASR is soybean.

63. The method of any one of embodiments 59-62, wherein said composition controls one or more fungal pathogens.

64. The method of embodiment 63, wherein the one or more fungal pathogens are selected from the group consisting of Botrytis cinerea, Cersospora spp, Cercospora sojina, Cercospora beticola, Alternaria solani, Rhizoctonia solani, Blumeria graminis f. sp. Tritici, Erysiphe necator, Podosphaera xanthii, Golovinomyces cichoracearum, Erysiphe lagerstroemiae, Sphaerotheca pannosa, Colletotrichum cereale, Apiognomonia errabunda, Apiognomonia veneta, Colletotrichum gloeosporiodes, Discula fraxinea, Plasmopara viticola, Pseudoperonospora cubensis, Peronospora belbahrii, Bremia lactucae, Peronospora lamii, Plasmopara obduscens, Pythium cryptoirregulare, Pythium aphanidermatum, Pythium irregulare, Pythium sylvaticum, Pythium myriotylum, Pythium ultimum, Phytophthora capsici, Phytophthora nicotianae, Phytophthora infestans, Phytophthora tropicalis, Phytophthora sojae, Fusarium graminearum, Fusarium solani, Fusarium oxysporurn, Fusarium graminicola, Gibberella zeae, Colletotrichum graminicola, Phakopsora sp., Phakopsora meibomiae, Phakopsora pachyrizi, Puccinia triticina, Puccinia recondita, Puccinia striiformis, Puccinia graminis, Puccinia spp., Venturia inaequalis, Verticillium spp, Erwinia amylovora, Monilinia fructicola, Monilinia lax, and Monilinia fructigena.

65. The method of embodiment 64, wherein said composition controls one or more fungal pathogens selected from the group consisting of Botrytis cinerea, Cercospora sojina, Alternaria solani, Rhizoctonia solani, Erysiphe necator, Podosphaera xanthii, Colletotrichum cereal, Plasmopara viticola, Peronospora belbahrii, Pythium aphanidermatum, Pythium sylvaticum, Pythium myriotylum, Pythium ultimum, Phytophthora nicotianae, Phytophthora infestans, Phytophthora tropicalis, Phytophthora sojae, Fusarium graminearum, Fusarium solani, Phakopsora pachyrizi, and Venturia inaequalisa.

66. The method of embodiment 64, wherein the one or more fungal pathogens comprise Phakopsora pachyrhizi or Phakopsora meibomiae.

67. The method of embodiment 66, wherein the one or more fungal pathogens comprise Phakopsora pachyrhizi.

68. The method of any one of embodiments 59-67, wherein said method further comprises applying an effective amount of a biocide, wherein said effective amount of the biocide selectively controls an organism of interest while not significantly damaging the crop.

69. The method of embodiment 68, wherein the bacterial strain or active variant thereof and the biocide are applied simultaneously.

70. The method of embodiment 68, wherein the bacterial strain or active variant thereof and the biocide are applied sequentially.

71. The method of any one of embodiments 68-70 where the biocide is a fungicide.

72. A method of making a modified bacterial strain comprising:
(a) providing a population of at least one bacterial strain comprising AIP27511, AIP35174, AIP25773, AIP15251, AIP61892, AIP79428, AIP14931, AIP39589, or AIP36895, or an active variant of any thereof, wherein the active variant comprises a bacterial strain having a genome within a Mash distance of about 0.015, wherein said bacterial strain is susceptible to a biocide of interest;
(b) culturing said bacterial strain in the presence of the biocide of interest; and,
(c) selecting a modified bacterial strain having an increased resistance to said biocide of interest.

73. The method of embodiment 72, where said culturing comprises increasing the concentration of the biocide over time.

74. The method of embodiment 72 or 73, where said biocide is glyphosate or glufosinate.

75. A method of treating or preventing a plant disease comprising applying to a plant having a plant disease or at risk of developing a plant disease an effective amount of:

(a) at least one of bacterial strain AIP27511, AIP35174, AIP25773, AIP15251, AIP61892, AIP79428, AIP14931, AIP39589, or AIP36895 or an active variant of any thereof, wherein the active variant comprises a bacterial strain having a genome within a Mash distance of about 0.015; and/or

(b) at least one of a spore or a forespore, or a combination of cells, forespores and/or spores from any one of AIP27511, AIP35174, AIP25773, AIP15251, AIP61892, AIP79428, AIP14931, AIP39589, or AIP36895 or an active variant of any thereof, wherein the active variant comprises a bacterial strain having a genome within a Mash distance of about 0.015; wherein said effective amount comprises at least about 10¹² to 10¹⁶ CFU per hectare, and wherein said bacterial strain controls a plant pathogen that causes the plant disease.

76. The method of embodiment 75, wherein the bacterial strain or active variant thereof treats or prevents one or more plant diseases.

77. The method of embodiment 76, wherein the one or more plant diseases comprise one or more fungal plant diseases.

78. The method of embodiment 77, wherein the one or more fungal plant diseases comprise Asian Soybean Rust (ASR).

79. The method of any one of embodiments 75-78, wherein the bacterial strain or active variant thereof controls one or more pathogens.

80. The method of embodiment 79, wherein the one or more pathogens comprise one or more fungal pathogens.

81. The method of embodiment 80, wherein the one or more fungal pathogens are selected from the group consisting of Botrytis cinerea, Cersospora spp, Cercospora sojina, Cercospora beticola, Alternaria solani, Rhizoctonia solani, Blumeria graminis f. sp. Tritici, Erysiphe necator, Podosphaera xanthii, Golovinomyces cichoracearum, Erysiphe lagerstroemiae, Sphaerotheca pannosa, Colletotrichum cereale, Apiognomonia errabunda, Apiognomonia veneta, Colletotrichum gloeosporiodes, Discula fraxinea, Plasmopara viticola, Pseudoperonospora cubensis, Peronospora belbahrii, Bremia lactucae, Peronospora lamii, Plasmopara obduscens, Pythium cryptoirregulare, Pythium aphanidermatum, Pythium irregulare, Pythium sylvaticum, Pythium myriotylum, Pythium ultimum, Phytophthora capsici, Phytophthora nicotianae, Phytophthora infestans, Phytophthora tropicalis, Phytophthora sojae, Fusarium graminearum, Fusarium solani, Fusarium oxysporurn, Fusarium graminicola, Gibberella zeae, Colletotrichum graminicola, Phakopsora sp., Phakopsora meibomiae, Phakopsora pachyrizi, Puccinia triticina, Puccinia recondita, Puccinia striiformis, Puccinia graminis, Puccinia spp., Venturia inaequalis, Verticillium spp, Erwinia amylovora, Monilinia fructicola, Monilinia lax, and Monilinia fructigena.

82. The method of embodiment 81, wherein said composition controls one or more fungal pathogens selected from the group consisting of Botrytis cinerea, Cercospora sojina, Alternaria solani, Rhizoctonia solani, Erysiphe necator, Podosphaera xanthii, Colletotrichum cereal, Plasmopara viticola, Peronospora belbahrii, Pythium aphanidermatum, Pythium sylvaticum, Pythium myriotylum, Pythium ultimum, Phytophthora nicotianae, Phytophthora infestans, Phytophthora tropicalis, Phytophthora sojae, Fusarium graminearum, Fusarium solani, Phakopsora pachyrizi, and Venturia inaequalisa.

83. The method of embodiment 81, wherein the one or more fungal pathogens comprise Phakopsora pachyrhizi or Phakopsora meibomiae.

84. The method of embodiment 83, wherein the one or more fungal pathogens comprise Phakopsora pachyrhizi.

The following examples are offered by way of illustration and not by way of limitation.

### EXAMPLES

### Example 1

### Materials and Methods

Plant Material:

The susceptible soybean cultivar Williams 82 was used in strain evaluation using the detached-leaf technique (Twizeyimana and Hartman, 2010) and using whole plant in growth chambers. Briefly, soybean seeds were sown in 18-cell plastic inserts that were filled with soil-less mix (Sunshine Mix, LC1; Sun Gro Horticulture Inc., Bellevue, Wash.) and placed inside a flat. Cells were fertilized at planting with slow-release pellets (Osmocote 19-6-12; 2 pellets per cm²). Flats were maintained inside a growth chamber (Percival Scientific, Inc., Boone, Iowa) maintained at 70% relative humidity (RH) with a daily cycle of 14 h of light and 10 h of darkness at 24 and 20° C., respectively.

Bacterial Strains:

Bacterial strains were plated on Luria Bertani medium or in liquid culture, CHA medium which consists of, per L, NaCl (5 g), tryptone (10 g), nutrient broth (8 g), CaCl₂ (0.14 mM), MgCl₂.6H₂O (0.2 mM), and MnCl₂.4H₂O (0.01 mM) and were purified to obtain single colonies. Single colonies were characterized morphologically or using molecular techniques.

Phakopsora pachyrhizi Isolate:

The isolate FL07-1 was used in all inoculations. The isolate is a single spore isolate obtained from infected soybean leaves collected from Gadseden County, Fla. in 2007.

### Strain Evaluation:

Evaluation on Detached-Leaf:

Briefly, leaf disks (3-cm diameter each) were sprayed with 120 μl of the bacterial strain (1×10⁸ spores/ml of water) of P. pachyrhizi. Leaf disks were inoculated with 120 μl spore suspension (1×10⁴ spores/ml of sterile distilled water) of P. pachyrhizi a day after application of the bacterial strain. Both the bacterial strain and P. pachyrhizi inoculation applications were done using an atomizer attached to an air compressor (Twizeyimana and Hartman, 2010). Leaf disks were placed adaxial side down on saturated 20×20 cm filter paper (Whatman International Ltd., Kent, England) in a plastic container (Blister Box 20×20 cm, Placon, Madison, Wis.); two filter papers were used per box. Boxes with leaf disks were incubated in the dark for a period of 12 h followed by a cycle of 13 hours of light (380 umol m-2s-1) and 11 h of darkness inside a tissue chamber (Percival Scientific, Inc.) maintained at 23° C. and 95% RH. Prior to incubation, boxes were placed inside zip bags (Webster Industries, Peabody, Mass.). The experimental design was a randomized complete block design with 2 replications and was repeated once.

Evaluation on Whole Plant:

Bacterial strains were selected from the initial screening (conducted using leaf disk) based on their uredinia counts and were evaluated on whole plant in growth chambers. In this evaluation, when plants were at V2-stage (Fehr et al. 1971), the first fully expanded trifoliate leaf was sprayed with the bacterial strain, and the inoculation with P. pachyrhizi was done a day after as described in detached-leaf evaluation. Sprayed plants were maintained in a growth chamber at 75% RH with a daily cycle of 14 h and 10 h of light and darkness at 22° C. and 24° C., respectively. The experimental design was a randomized complete block design with 3 replications and was repeated once.

### Data Collection and Results:

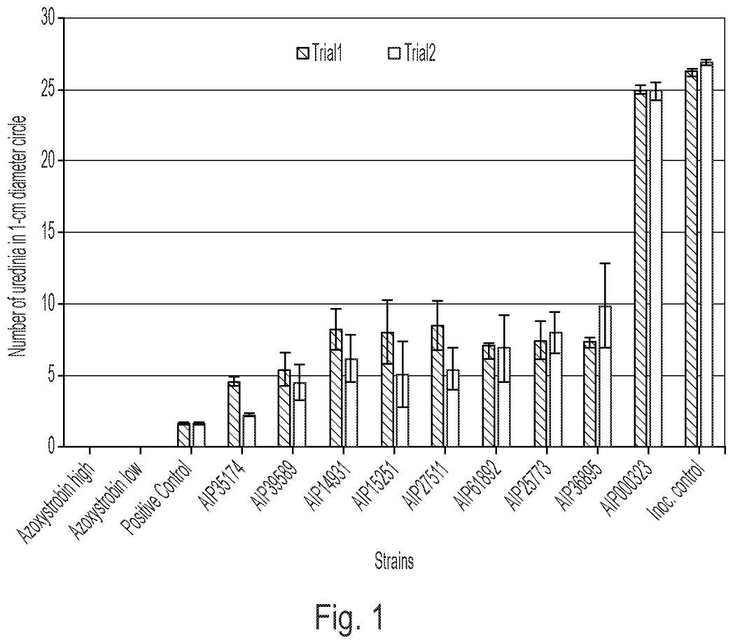

In both evaluations, data recorded were numbers of uredinia (uredinia counts) in 1-cm diameter circle recorded 14 days after inoculation. Nine bacterial strains that had <10 uredinia in 1-cm diameter circle were selected after evaluation on whole plant in growth chamber to be tested in the field (FIG. 1 & Table 2).

TABLE 2
Nine bacterial strains selected from evaluation
on whole plant in growth chambers

| # | AIP | Strains |
|---|---|---|
| 1 | 14931 | Bacillus thuringiensis |
| 2 | 15251 | Bacillus frigoritolerans |
| 3 | 25773 | Bacillus flexus |
| 4 | 27511 | Bacillus drentensis |
| 5 | 35174 | Bacillus cereus |
| 6 | 36895 | Bacillus simplex |
| 7 | 39589 | Bacillus acidiceler |
| 8 | 61892 | Bacillus subtilis subsp. Subtilis |
| 9 | 79428 | Burkholderia vietnamiensis |

### Example 2. Methods of Culturing

Bacterial strains were cultured in CHA media which consists of, per L, NaCl (5 g), tryptone (10 g), nutrient broth (8 g), CaCl₂ (0.14 mM), MgCl₂.6H₂O (0.2 mM), and MnCl₂.4H₂O (0.01 mM). Table 3 summarizes the incubation time, the concentration of bacteria (CFU/ml) achieved and percentage of sporulation.

TABLE 3

| | | Incubation | Concentration | |
|---|---|---|---|---|
| Strain | Medium | time (hrs) | (CFU/ml) | Sporulation |
| AIP23364 | CHA | 40 | 5e9 | Not tested |
| AIP 27511 | CHA | 50 | 1.25e9 | 50% |
| AIP 35174 | CHA | 50 | 1e9 | 80% |
| AIP 25773 | CHA | 50 | 8.3e8 | 100% |
| AIP 15251 | CHA | 50 | 8.4e8 | Did not |
| | | | | sporulate |
| AIP 61892 | CHA | 50 | 1.3e9 | 90% |
| AIP 79428 | CHA | 50 | 5e9 | Did not |
| | | | | sporulate |
| AIP 14931 | CHA | 50 | 2e8 | 50% |
| AIP 39589 | CHA | 46.5 | 8.7e8 | ²100% |
| | | | | intermediate |
| AIP 36895 | CHA | 48 | 6.2e8 | Did not |
| | | | | sporulate |

²Formation of forespore, did not form endospore

### Example 3. Field Trials for the Various Bacterial Strains or Active Variants Thereof

The various bacterial strains recited in Table 2 are applied to soybeans in the field. Treatments are applied at 16.8 Gallons/Acre with treatments applied to achieve uniform plant coverage per general treatment guidelines for ASR treatment. The first treatment is applied at R1 with a follow up treatment applied at 14 days and 28 days after first treatment. The specific treatments are outlined below.

### Treatments:

1. Untreated Check
2. Inoculated Check
3. Quadris at 6.2 oz/acre
4. Quadris at 2.1 oz/acre
5. AIP27511 at 7.5 g/L
6. AIP35174 at 7.5 g/L
7. AIP25773 at 7.5 g/L
8. AIP15251 at 7.5 g/L
9. AIP61892 at 7.5 g/L
10. AIP79428 at 7.5 g/L
11. AIP14931 at 7.5 g/L
12. AIP39589 at 7.5 g/L
13. AIP36895 at 7.5 g/L

### Example 4. Field Trials for the Various Bacterial Strains or Active Variants Thereof

The various bacterial strains recited in Table 2 are applied to soybeans in the field. Bacterial strain treatments are applied at 20 Gallons/Acre with treatments applied to achieve uniform plant coverage per general treatment guidelines for ASR treatment. The first treatment is applied at R1 with a follow up treatment applied at 14 days after first treatment. The specific treatments are outlined below.

### Treatments:

1. Untreated Check
2. Inoculated Check
3. Quadris at 6.2 oz/acre
4. Quadris at 2.1 oz/acre
5. AIP27511 at 7.5 g/L
6. AIP35174 at 7.5 g/L
7. AIP25773 at 7.5 g/L
8. AIP15251 at 7.5 g/L
9. AIP61892 at 7.5 g/L
10. AIP79428 at 7.5 g/L
11. AIP14931 at 7.5 g/L
12. AIP39589 at 7.5 g/L
13. AIP36895 at 7.5 g/L

### Example 5. Rhizoctonia Damping-Off Assay on Soybean Mock Seed Treatment/in-Furrow

11-14 day old Rhizoctonia solani infested grain was ground. The ground inoculum was screed through a #10 screen to remove any grain that was not ground well. The ground, screened infected grains was added to Fafard Superfine Germination media at 1.5 grams of ground inoculum to 1 liter of soil mix by volume. Germination mix, inoculum, and 1 liter of water per 75 liters of germination media was added to a cement mixer and mixed until everything was well incorporated. The well incorporated media-inoculum material was placed into a secondary holding container with a lid and held at 20° C. for 18 hours before using in the assay.

606-cell planting trays were filled with inoculated germination media making sure to not pack the media too firmly. One soybean seed was sown per 606 cell, planting at a depth of 1.5 to 2 cm leaving the planting holes open if applying treatments as a liquid formulation. Individual planting cells were treated with one of the re-suspended strains set forth in Table 1 at 3 ml per cell/seed. The seed treatment was directly over the top of the seed. Once treatments were applied, the shake flats were shaken lightly to close planting holes. The planting trays were lightly watered and placed in a humidity dome on the flat. After 3-4 days flats were checked for moisture and lightly watered as needed to ensure cells were evenly moist. The humidity dome was replaced after watering.

Data Collection and Results:

After 10-12 days, the assay was evaluated to determine the number of seeds that germinated. Data are reported as the % of seeds that germinated out of a total of 6 seeds per treatment. Eight strains with germination rates ≥50% and comparable to the non-inoculated control were selected for field testing (Table 4).

TABLE 4
Bacterial strains with activity against R. solani
in the soybean seed germination assay.

| | % | Number of |
|---|---|---|
| AIP | germinated | Reps |
| AIP061892 | 64 | 8 |
| AIP079428 | 58 | 7 |
| Non-inoculated | 86 | 22 |
| Inoculated | 27 | 22 |

### Example 6. Methods of Formulation for the Various Bacterial Strains

The culture produced in Example 2 was centrifuged, 20 minutes, 10,000 rpm to produce a pellet. The supernatant was poured off and another volume of culture fluid was added to the previous pellet and centrifuged again (this was to reduce the number of centrifuge tubes required for each harvest).

End product material was produced by adding 5% (by mass of pellet) of glycerol to the cell pellet and then mixed with a spatula. 20% (by mass) of Microcel E was transferred to a food processor and the glycerol/pellet was poured over the microcell. This material was blended using the knife blade attachment of the food processor for not more than 10 seconds. The product was dried overnight at 40° C. to approximately an a_{w} of 0.3.

### Example 7. Greenhouse Evaluation of Bacterial Strains Against Asian Soybean Rust

Plant Material and Bacterial Strains:

Cultivar Williams 82 was used in these experiments. The bacterial strains were prepared as follows. Fermentation culture broth was spun down and the pellet mass was weighed. For each 100 g of pellet material, 5 g of glycerol was added (5% of the pellet mass). Glycerol was mixed by hand until a uniform consistency is achieved. A total of 20 g (20% by weight of cell paste) of microcell-E (Tmery's Celite) was added to a food processor equipped with a Sabatier blade. Cell paste, glycerol, and micro-cell were homogenized briefly into a partially dry, crumb-like structure. This end product was spread into aluminum trays and dried at 40° C., overnight. Once the product dryness reached a water activity of 0.3 or less, it was milled and screened and was stored at 4° C.

Phakopsora pachyrhizi Isolate:

The isolate FL07-1 described above was used in experiments in Illinois and mixtures of spores collected in 2014 and 2015 were used in experiments in Florida.

Greenhouse Strain Evaluation:

In Illinois, two trials were conducted in a biosafety level 2 greenhouse. The conditions in the greenhouse were set for 22±2° C. under a 16-h photoperiod with supplemental illumination provided by 1,000-W Metalarc high-intensity lamps (Sylvania, Danvers, Mass.). Seeds of Williams 82 were sown in soil-less mix (Sunshine Mix, LC1; Sun Gro Horticulture Inc.) in 5-inch pots, and fertilized at planting with slow-release pellets (Osmocote 19-6-12; 1 to 2 pellets per cm²). Plants were thinned to one plant per pot after emergences and the experimental design was a randomized complete block design with 4 replicates for each treatment. Plants were sprayed with strains according to the protocol at growth stage V2-stage (Fehr et al. 1971), and they were inoculated with a spore suspension of isolate FL07-1 (1×10⁵ spores/ml of sterile distilled water) until runoff using a hand sprayer a day after strain application. Inoculated plants were left in mist chamber overnight and then were moved to greenhouse bench for symptom development. Fourteen days later, plants were retreated according to protocol. Six days after the second treatment, rust severity data was recorded.

In Florida, seeds of Williams 82 were sown into 22.8-cm-diameter plastic pots containing Metro Mix 300 (Sun GroHorticultural Distributors Inc., Bellevue, Wash.). Plants were maintained in a rust-free glass green-house on metal benches at an average temperature of 26° C. and an average relative humidity of 61%. Plants were thinned to one plant per pot after emergences. The experimental design was a randomized complete block design with 3 replicates for each treatment. Plants were sprayed with strains according to the protocol at growth stage R1-stage (Fehr et al. 1971), and were inoculated a day later using a mixture of P. pachyrhizi. Strain treatments were reapplied 14 days after the first treatments. Rust severity was recorded when plants were at R4- or 5-stage.

Data Collection:

In Illinois, rust severity was scored by counting the number of sporulating uredinia in an arbitrarily selected 1-cm diameter circle from each leaflet of inoculated trifoliate leaves. The data is shown in Table 5. In Florida, the data recorded were percent soybean rust severity from a randomly selected plant. The data is shown in Table 6.

TABLE 5
Number of sporulating uredinia per 1-cm diameter circle of leaf
tissue treated with different bacterial strains in the greenhouse
experiment conducted in Illinois. A (—) indicates “not
tested”. Samples represented by a different letter (eg., a,
ab, b, c, cd, d, e) had statistically significant different values.

Number of sporulating uredinia
per 1-cm dia circle

| Strain | Trial 1 | Trial 2 |
|---|---|---|
| AIP039589 | 4.2 b | 3.5 ab |
| AIP027511 | — | — |
| AIP035174 | 3.0 ab | 17.8 d |
| AIP025773 | 10.3 | — |
| AIP015251 | 7.0 c | 15.2 d |
| AIP061892 | 5.1 b | 3.6 ab |
| AIP079428 | — | 4.2 b |
| AIP014931 | 7.8 c | 24.1 e |
| AIP036895 | — | — |
| Fungicide (Quadris) | 0 a | 0 a |
| Inoculated Control | 12.7 d | 28.5 e |

TABLE 6
Percent rust severity of soybean plants treated with different
bacterial strains in the greenhouse experiment conducted
in Florida. A (—) indicates “not tested”. Samples
represented by a different letter (eg., a, ab, b, c, cd,
d, e) had statistically significant different values.

| Strain | % Average rust severity |
|---|---|
| AIP039589 | 13.3 b |
| AIP027511 | — |
| AIP035174 | 17.1 bc |
| AIP025773 | 15.9 bc |
| AIP015251 | 21.0 d |
| AIP061892 | 11.0 b |
| AIP079428 | — |
| AIP014931 | 23.8 d |
| AIP036895 | — |
| Fungicide (Quadris) | 1.3 a |
| Inoculated Control | 22.4 d |

### Example 8. Field Evaluations Against Asian Soybean Rust in Florida

A field experiment was conducted in Florida in 2015 at the North Florida Research and Education Center in Quincy. The average monthly temperature from July to September during evaluation, ranged from 24 to 27° C. The plant material, bacterial strains and P. pachyrhizi isolate were similar to those described in the greenhouse experiment in Florida (Example 7). Plants were sprayed with strains according to the protocol at R1 growth stage (Fehr et al. 1971) and were inoculated a day after using a mixture of P. pachyrhizi that was followed by subsequent natural infection. Strain treatments were reapplied at 14 days after the first treatment.

Percent soybean rust severity was recorded from a randomly selected plant at R5-stage. The data is shown in Table 7.

TABLE 7
Percent rust severity of soybean plants treated with different bacterial
strains in the field experiment conducted in Florida. A (—)
indicates “not tested”. Samples represented by a different
letter (eg., a, ab) had statistically significant different values.

| Strains | % Average rust severity |
|---|---|
| AIP039589 | — |
| AIP027511 | — |
| AIP035174 | 6.4 ab |
| AIP025773 | 3.4 a |
| AIP015251 | 3.9 a |
| AIP061892 | 3.6 a |
| AIP079428 | — |
| AIP014931 | 2.9 a |
| AIP036895 | — |
| Fungicide (Quadris) | 2.5 a |
| Inoculated Control | 5.8 ab |

### Example 9. Rhizoctonia Damping-Off Assay—Soybean Mock Seed Treatment/in-Furrow

11-14 day old Rhizoctonia solani infested grain is ground. The ground inoculum is screened through a #10 screen to remove any grain that is not ground well. The ground, screened infected grains are added to Fafard Superfine Germination media at 1.5 grams of ground inoculum to 1 liter of soil mix by volume. Germination mix, inoculum, and 1 liter of water per 75 liters of germination media are added to a cement mixer and mix until everything is well incorporated. The well incorporated media-inoculum material is placed into a secondary holding container with a lid and held at 20° C. for 18 hours before using in the assay.

606-cell planting trays are filled with inoculated germination media making sure to not pack the media too firmly. One soybean seed is sown per 606 cell, planting at a depth of 1.5 to 2 cm leaving the planting holes open if applying treatments as a liquid formulation. Individual planting cells are treated with one of the re-suspended strains set forth in Table 2 at 3 ml per cell/seed. The seed treatment is directly over the top of the seed. Once treatments is applied, the shake flats is shaken lightly shake to close planting holes. The planting trays are lightly watered and placed in a humidity dome on the flat. After 3-4 days, flats are checked for moisture and lightly watered as needed to ensure cells are evenly moist. The humidity dome is replaced after watering.

Data Collection and Results:

After 10-12 days, the assay is evaluated to determine the number of seeds that germinated. Data is reported as the % of seeds that germinated out of a total of 6 seeds per treatment.

### Example 10. Field Trials Against Various Fungal Pathogens for the Various Bacterial Strains

The various bacterial strains recited in Table 2 are applied to the crops listed in Table 8 in the field under the current agronomic practices at listed in Table 8 to achieve uniform plant coverage and follow proper agronomic practices. Treatments are applied preventatively and/or curatively at the appropriate timings per disease.

TABLE 8

| | | | Treatment | Treatment | Application |
|---|---|---|---|---|---|
| Crop | Pathogen | Rate | Volume | Number | Interval/Timing |
| All crops | Gray Mold | 5 g/L | 25-200 | 1 to 10 | 7 to 14 days |
| | | | Gallons/Acre | | |
| Ornamental | Cercospora | 5 g/L | 100-300 | 1 to 4 | 7 to 14 days |
| Crops | Leaf Spots | | Gallons/Acre | | |
| Soybean | Cercospora | 5 g/L | 5-20 | 1 to 3 | V7, R1, R3, R5 |
| | Leaf Spots | | Gallons/Acre | | |
| Beet, Spinach, | Cercospora | 5 g/L | 15-50 | 3 to 6 | 7 to 14 days |
| Chard | Leaf Spots | | Gallons/Acre | | |
| Solanaceous | Early Blight | 5 g/L | 15-50 | 4 to 10 | 7 to 14 days |
| Crops | | | Gallons/Acre | | |
| Grape | Powdery | 5 g/L | 15-50 | 3 to 8 | 7 to 14 days |
| | Mildew | | Gallons/Acre | | |
| Cucurbit | Powdery | 5 g/L | | 2 to 8 | 7 to 14 days |
| | Mildew | | | | |
| Turf/other | Anthrancose | 5 g/L | 87-120 | 2 to 6 | 7 to 14 days |
| grasses | leaf spot | | Gallons/Acre | | |
| Grape | Downy | 5 g/L | 50-100 | 2 to 6 | 7 to 14 days |
| | Mildew | | Gallons/Acre | | |
| Leafy Greens | Downy | 5 g/L | 25 to 75 | 2 to 6 | 7 to 14 days |
| | Mildew | | Gallons/Acre | | |
| Basil | Downy | 5 g/L | 25-75 | 2 to 6 | 7 to 14 days |
| | Mildew | | Gallons/Acre | | |
| Ornamental | Late Blight | 5 g/L | 100-300 | 2 to 6 | 7 to 14 days |
| Plants | | | Gallons/Acre | | |
| Cucurbit/Peppers | Late Blight | 5 g/L | 25-100 | 2 to 10 | 7 to 14 days |
| | | | Gallons/Acre | | |
| Solanaceous | Late Blight | 5 g/L | 25-100 | 2 to 10 | 7 to 14 days |
| Crops | | | Gallons/Acre | | |
| Soybean | Late Blight | 5 g/L | 5-20 | 1 to 3 | V4 to R5 |
| | | | Gallons/Acre | | |
| Soybean | Rust | 5 g/L | 5-20 | 1 to 4 | V4 to R5 |
| | | | Gallons/Acre | | |
| Rosacea family | Fire Blight | 5 g/L | 20-100 | 1 to 3 | Pre/Post Flower |
| | | | Gallons/Acre | | |
| Malus | Apple Scab | 5 g/L | 20-100 | 1 to 5 | 7 to 14 days |
| | | | Gallons/Acre | | |
| Stone Fruits | Brown Rot | 5 g/L | 20-100 | 1 to 3 | Pre/Post Flower and |
| | | | Gallons/Acre | | Fruit Set |
| Rice | Sheath | 5 g/L | 5-20 | 1 to 3 | Prior to Canopy |
| | Blight | | Gallons/Acre | | Closure |
| Cereals | Fusarium | 5 g/L | 5-20 | 1 to 2 | Feekes 7, 9, and/or |
| | Head Blight | | Gallons/Acre | | 10.51 |

The specific treatments are outlined below:

### Foliar Pathogen Treatment List: Early Blight

6-10 treatments
Treatment Volume: 100 gallons/acre

### Treatment List:

1. Non-Inoculated, untreated Check
2. Inoculated Check
3. Chemical control chosen by cooperator applied at label instructions
4. Biological control Serenade applied at label instructions
5. Experimental Biological Foliar treatment(s) at 5 g/L plus Capsil at 3 oz/100 gallons

### Example 11. Field Trials Against Various Fungal Pathogens for the Various Bacterial Strains or Active Variants Thereof Employing Seed Treatments

The various bacterial strains recited in Table 2 are applied to the crops listed in Table 9 as seed treatments prior to being planted into the field. Bacterial strain treatments are applied for preventative control of the diseases and at the application rates in Table 10. The specific treatments are outlined below.

TABLE 9

| Soybean | Canola | Wheat | Cereal Grains |
|---|---|---|---|
| Maize | Cucurbit | Cotton | Solanaceous Crops |
| Beets | Leafy Greens | Verticillium Whilt | Sunflower oil and |
| | | | seed |

### Seed Treatment Trial Treatment List:

1. Non-inoculated Check
2. Inoculated Check
3. Disease appropriate Seed Treatment Chemical Check chosen and applied by cooperator
5. Biological Experimental Seed Treatment(s)

TABLE 10

| | | | Treatment |
|---|---|---|---|
| Crop | Pathogen | Rate | Type |
| Row Crops/Vegetables | Pythium | 10e4 to 10e12 | Seed Treatment |
| Row Crops/Vegetables | Phytophthora | 10e4 to 10e12 | Seed Treatment |
| Row Crops/Vegetables | Fusarium Wilt | 10e4 to 10e12 | Seed Treatment |
| Row Crops/Vegetables | Soybean Death | 10e4 to 10e12 | Seed Treatment |
| | Syndrome | | |
| Row Crops/Vegetables | Rhizoctonia | 10e4 to 10e12 | Seed Treatment |
| | solani | | |
| Row Crops/Vegetables | Verticillium | 10e4 to 10e12 | Seed Treatment |
| | Wilt | | |
| Row Crops/Vegetables | Corn Stalk Rot | 10e4 to 10e12 | Seed Treatment |

### Example 12. Field Trials Against Various Fungal Pathogens for the Various Bacterial Strains or Active Variants Thereof Employing in-Furrow Treatments

The various bacterial strains or active variants thereof recited in Table 1 are applied to the crops listed in Table 9 as in-furrow treatments at time of planting as preventative control for the diseases and at the treatment rates listed in Table 11. The specific treatments are outlined below:

### In-Furrow Trial Treatment List:

1. Non-inoculated Check
2. Inoculated Check
3. In-Furrow Biological Treatment(s) 5 g/L+Capsil at 6 oz/100 Gallons at 15 Gallons/Acre
4. Disease appropriate In-Furrow Chemical Check as chosen and applied by cooperator.

TABLE 11

| Crop | Pathogen | Rate | Treatment/Volume |
|---|---|---|---|
| Row Crops/Vegetables | Pythium | 5 g/L | 2 to 15 Gallons/Acre |
| Row Crops/Vegetables | Phytophthora | 5 g/L | 2 to 15 Gallons/Acre |
| Row Crops/Vegetables | Fusarium Wilt | 5 g/L | 2 to 15 Gallons/Acre |
| Row Crops/Vegetables | Soybean Death | 5 g/L | 2 to 15 Gallons/Acre |
| | Syndrome | | |
| Row Crops/Vegetables | Rhizoctonia | 5 g/L | 2 to 15 Gallons/Acre |
| | solani | | |
| Row Crops/Vegetables | Verticillium Wilt | 5 g/L | 2 to 15 Gallons/Acre |
| Row Crops/Vegetables | Corn Stalk Rot | 5 g/L | 2 to 15 Gallons/Acre |

### Example 13—Biological Control Strain Seed Treatment Protocol

The seed treatment formulation was made by mixing 10 g formulated strain plus 30 ml water plus 15 ml Unicoat Polymer. The weighed out seed is placed in a sterilized mason jar. An appropriate amount of seed treatment solution based off of seed weight (0.05 ml/25 g seed), the mixture is shaken for 60 seconds or until the seeds were visually well coated. The seeds are placed into a single layer in a foil roasting pan and placed under a laminar flow hood for 1 hour or until seeds are dry. Once the seeds dry, they are placed in an air tight container and stored at RT.

### Example 14. Wettable Powder Formulations

One hundred grams of cell paste from each of the strains denoted below in Table 12 was mixed with 5 g of glycerol and 20 g of synthetic calcium silicate using a food processor. This material was dried at 40° C. to a water activity of less than 0.30 at which time it contained CFU/g as noted in Table 12. The dried powder formulation was stored in vacuum sealed mylar pouches at 22 C. The dried powder formulation retained antifungal activity.

TABLE 12

| Strain Name | CFU/g wettable powder |
|---|---|
| AIP14931 | 1.16 × 10¹¹ |
| AIP15251 | 1.47 × 10¹¹ |
| AIP25773 | 6.90 × 10¹⁰ |
| AIP35174 | 1.23 × 10¹¹ |
| AIP61892 | 6 × 10¹⁰ |
| AIP79428 | 4.73 × 10⁹ |

### Example 15. Pythium Field Trials

The bacterial strains AIP061892 and AIP079428 were applied as seed treatments to Soybean variety W3103. The bacterial strains were each formulated as a wettable powder as described in Example 14 and then turned into seed treatments by combining 10 g of formulated bacterial strain with 30 ml water and 15 ml Seed Coating Polymer (Unicoat) and then shaking until a uniform solution was made. The finished solution was applied to 1 kg of soybean seed and allowed to dry under a laminar flow hood for 12 hours

Pythium inoculum was grown on millet grain and applied via in-furrow application at 1.25 g/ft and was applied at planting with treated soybeans seeded at 130,000 seeds per acre on day 1. Whole row stand counts were taken 17 days later. The specific treatments are outlined below.

### Treatments:

1. Untreated Check
2. Inoculated Check
3. Quadris at 0.4 fluid ounces/Acre
4. AIP061892 Seed Treatment
5. AIP079428 Seed Treatment

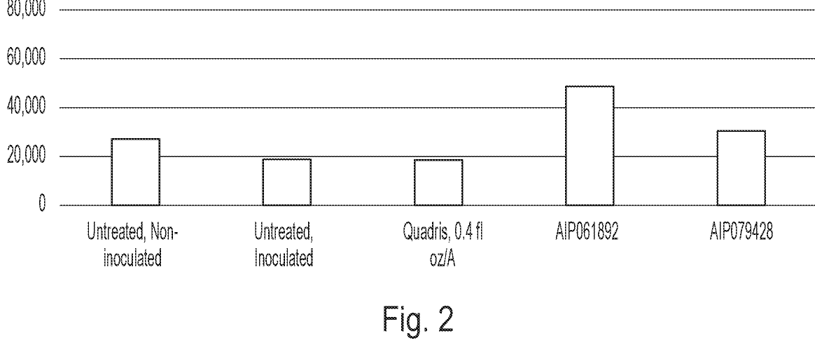

FIG. 2 shows the number of germinated seedlings (stand count) per acre. FIG. 2 demonstrates that AIP061892 and AIP079428 each produced about a 2-fold increase in germination over inoculated control.

### Example 16. Rhizoctonia solani Field Trials

The bacterial strains AIP061892 and AIP079428 were applied as seed treatments to Soybean variety W3103. The bacterial strains were each formulated as a wettable powder as noted in Example 14 and then turned into seed treatments by combining 10 g of formulated bacterial strain with 30 ml water and 15 ml Seed Coating Polymer (Unicoat) and then shaking until a uniform solution was made. The finished solution was applied to 1 kg of soybean seed and allowed to dry under a laminar flow hood for 12 hours.

Rhizoctonia solani inoculum was grown on sorghum grain and applied via in-furrow application at 1.25 g/ft and was applied at planting with treated soybeans seeded at 130,000 seeds per acre on day 1. Whole row stand counts were taken 17 days later. The specific treatments are outlined below:

### Treatments:

1. Untreated Check
2. Inoculated Check
3. Quadris at 0.4 fluid ounces/Acre
4. AIP061892 Seed Treatment
5. AIP079428 Seed Treatment

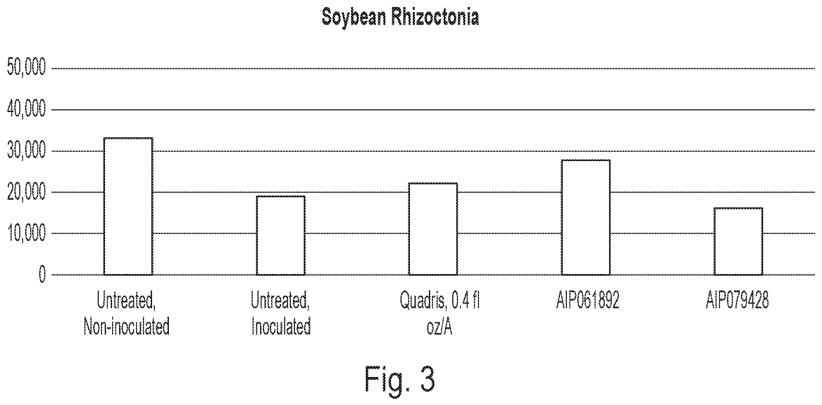

FIG. 3 shows the number of germinated seedlings (stand count) per acre. FIG. 3 demonstrates that AIP061892 produced a 50% recovery in germination over inoculated control.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. (canceled)

2. A formulation comprising a biocontrol agent NRRL No. B-67089, NRRL No. B-67082, NRRL No. B-67084, NRRL No. B-67085, NRRL No. B-67083, NRRL No. B-67081, NRRL No. B-67088, NRRL No. B-67087, or NRRL No. B-67086, or an active variant of any thereof having a genome within a Mash distance of about 0.015 to the biocontrol agent, wherein said formulation is a stable dry formulation or a stable liquid formulation.

3. The formulation of claim 2, wherein the biocontrol agent further comprises at least one of a spore, or a forespore, or a combination of cells, forespores and/or spores from NRRL No. B-67089, NRRL No. B-67082, NRRL No. B-67084, NRRL No. B-67085, NRRL No. B-67083, NRRL No. B-67081, NRRL No. B-67088, NRRL No. B-67087, or NRRL No. B-67086, or an active variant of any thereof having a genome within a Mash distance of about 0.015 to the biocontrol agent.

4. The formulation of claim 2, wherein said stable dry formulation is dried to a water activity of 0.3 or less.

5. The formulation of claim 2, wherein the formulation is a spray dried formulation, a wettable powder or a granule.

6. The formulation of claim 2, wherein the formulation further comprises a pesticide, a fungicide, an insecticide or a herbicide.

7. The formulation of claim 6, wherein the fungicide comprises aliphatic nitrogen fungicides, amide fungicides, acylamino acid fungicides, anilide fungicides, benzanilide fungicides, furanilide fungicides, sulfonanilide fungicides, benzamide fungicides, furamide fungicides, phenylsulfamide fungicides, sulfonamide fungicides, valinamide fungicides, antibiotic fungicides, strobilurin fungicides, methoxyacrylate strobilurin fungicides, methoxycarbanilate strobilurin fungicides, methoxyiminoacetamide strobilurin fungicides, methoxyiminoacetate strobilurin fungicides, aromatic fungicides, arsenical fungicides, aryl phenyl ketone fungicides, benzimidazole fungicides, benzimidazole precursor fungicides, benzothiazole fungicides, botanical fungicides, bridged diphenyl fungicides, carbamate fungicides, benzimidazolylcarbamate fungicides, carbanilate fungicides, conazole fungicides, conazole fungicides (imidazoles), conazole fungicides (triazoles), copper fungicides, cyanoacrylate fungicides, dicarboximide fungicides, dichlorophenyl dicarboximide fungicides, phthalimide fungicides, dinitrophenol fungicides, dithiocarbamate fungicides, cyclic dithiocarbamate fungicides, polymeric dithiocarbamate fungicides, dithiolane fungicides, fumigant fungicides, hydrazide fungicides, imidazole fungicides, inorganic fungicides, mercury fungicides; inorganic mercury fungicides, organomercury fungicides, morpholine fungicides, organophosphorus fungicides, organotin fungicides, oxathiin fungicides, oxazole fungicides, polysulfide fungicides, pyrazole fungicides, pyridine fungicides, pyrimidine fungicides, anilinopyrimidine fungicides, pyrrole fungicides, quaternary ammonium fungicides, quinoline fungicides, quinone fungicides, quinoxaline fungicides, thiadiazole fungicides, thiazole fungicides, thiazolidine fungicides, thiocarbamate fungicides, thiophene fungicides, triazine fungicides, triazole fungicides, triazolopyrimidine fungicides, urea fungicides, zinc fungicides, unclassified fungicides, or mefenoxam.

8. The formulation of claim 6, wherein the fungicide comprises prothioconazole, azoxystrobin, fluopicolide, chlorothalonil, fosetyl, fenhexamid, flutriafol, difenoconazole, tebuconazole, tetraconazole, pyraclostrobin, trifloxystrobin, propiconazole, fluoxastrobin, flutolanil, metconazole, or metrafenone.

9. A coated seed comprising a seed and a coating on the seed, wherein the coating comprises a formulation comprising a biocontrol agent NRRL No. B-67089, NRRL No. B-67082, NRRL No. B-67084, NRRL No. B-67085, NRRL No. B-67083, NRRL No. B-67081, NRRL No. B-67088, NRRL No. B-67087, or NRRL No. B-67086, or an active variant of any thereof having a genome within a Mash distance of about 0.015 to the biocontrol agent.

10. The coated seed of claim 9, wherein the biocontrol agent further comprises at least one of a spore, or a forespore, or a combination of cells, forespores and/or spores from NRRL No. B-67089, NRRL No. B-67082, NRRL No. B-67084, NRRL No. B-67085, NRRL No. B-67083, NRRL No. B-67081, NRRL No. B-67088, NRRL No. B-67087, or NRRL No. B-67086, or an active variant of any thereof having a genome within a Mash distance of about 0.015 to the biocontrol agent.

11. The coated seed of claim 9, wherein the seed is from a monocot.

12. The coated seed of claim 11, wherein the monocot is corn, sorghum, wheat, rice, sugarcane, barley, oats, rye, millet, coconut, pineapple or banana.

13. The coated seed of claim 9, wherein the seed is from a dicot.

14. The coated seed of claim 13, wherein the dicot is sunflower, tomato, peppers, potato, cotton, soybean, sugarbeet, tobacco, sweet potato, alfalfa, safflower, peanuts, cassava, coffee, cocoa, cucumber, lettuce, olive, peas, or tea.

15. The coated seed of claim 9, wherein the coating further comprises a pesticide, a fungicide, an insecticide or a herbicide.

16. The coated seed of claim 15, wherein the fungicide comprises aliphatic nitrogen fungicides, amide fungicides, acylamino acid fungicides, anilide fungicides, benzanilide fungicides, furanilide fungicides, sulfonanilide fungicides, benzamide fungicides, furamide fungicides, phenylsulfamide fungicides, sulfonamide fungicides, valinamide fungicides, antibiotic fungicides, strobilurin fungicides, methoxyacrylate strobilurin fungicides, methoxycarbanilate strobilurin fungicides, methoxyiminoacetamide strobilurin fungicides, methoxyiminoacetate strobilurin fungicides, aromatic fungicides, arsenical fungicides, aryl phenyl ketone fungicides, benzimidazole fungicides, benzimidazole precursor fungicides, benzothiazole fungicides, botanical fungicides, bridged diphenyl fungicides, carbamate fungicides, benzimidazolylcarbamate fungicides, carbanilate fungicides, conazole fungicides, conazole fungicides (imidazoles), conazole fungicides (triazoles), copper fungicides, cyanoacrylate fungicides, dicarboximide fungicides, dichlorophenyl dicarboximide fungicides, phthalimide fungicides, dinitrophenol fungicides, dithiocarbamate fungicides, cyclic dithiocarbamate fungicides, polymeric dithiocarbamate fungicides, dithiolane fungicides, fumigant fungicides, hydrazide fungicides, imidazole fungicides, inorganic fungicides, mercury fungicides; inorganic mercury fungicides, organomercury fungicides, morpholine fungicides, organophosphorus fungicides, organotin fungicides, oxathiin fungicides, oxazole fungicides, polysulfide fungicides, pyrazole fungicides, pyridine fungicides, pyrimidine fungicides, anilinopyrimidine fungicides, pyrrole fungicides, quaternary ammonium fungicides, quinoline fungicides, quinone fungicides, quinoxaline fungicides, thiadiazole fungicides, thiazole fungicides, thiazolidine fungicides, thiocarbamate fungicides, thiophene fungicides, triazine fungicides, triazole fungicides, triazolopyrimidine fungicides, urea fungicides, zinc fungicides, unclassified fungicides, or mefenoxam.

17. The coated seed of claim 15, wherein the fungicide comprises prothioconazole, azoxystrobin, fluopicolide, chlorothalonil, fosetyl, fenhexamid, flutriafol, difenoconazole, tebuconazole, tetraconazole, pyraclostrobin, trifloxystrobin, propiconazole, fluoxastrobin, flutolanil, metconazole, or metrafenone.

18. A composition comprising an effective amount of (a) a biocontrol agent NRRL No. B-67089, NRRL No. B-67082, NRRL No. B-67084, NRRL No. B-67085, NRRL No. B-67083, NRRL No. B-67081, NRRL No. B-67088, NRRL No. B-67087, or NRRL No. B-67086, or an active variant of any thereof having a genome within a Mash distance of about 0.015 to the biocontrol agent; and, (b) a pesticide, a fungicide, an insecticide, or a herbicide; wherein said composition controls a plant pathogen.

19. The composition of claim 18, wherein the biocontrol agent is present in about 10⁵ CFU/gram to about 10¹² CFU/gram or in about 10⁵ CFU/ml to about 10¹² CFU/ml.

20. A method of controlling a plant pathogen comprising applying to a plant, a seed or an area of cultivation an effective amount of a biocontrol agent NRRL No. B-67089, NRRL No. B-67082, NRRL No. B-67084, NRRL No. B-67085, NRRL No. B-67083, NRRL No. B-67081, NRRL No. B-67088, NRRL No. B-67087, or NRRL No. B-67086, or an active variant of any thereof having a genome within a Mash distance of about 0.015, wherein said effective amount controls said plant pathogen.

21. A method of growing a plant comprising applying to a plant, a seed or an area of cultivation an effective amount of a biocontrol agent NRRL No. B-67089, NRRL No. B-67082, NRRL No. B-67084, NRRL No. B-67085, NRRL No. B-67083, NRRL No. B-67081, NRRL No. B-67088, NRRL No. B-67087, or NRRL No. B-67086, or an active variant of any thereof having a genome within a Mash distance of about 0.015 to the biocontrol agent, wherein said effective amount controls a plant pathogen.
